(19) United States
(12) Patent Application Publication
(10) Pub. No.: US 2025/0093362 A1
(43) Pub. Date: Mar. 20, 2025

(54) HIGH-THROUGHPUT ENGINEERING OF MOLECULAR GLUES

(71) Applicant: Frontier Medicines Corporation, South San Francisco, CA (US)

(72) Inventors: Daniel A. ERLANSON, San Francisco, CA (US); Kevin Richard WEBSTER, San Mateo, CA (US)

(21) Appl. No.: 18/294,114

(22) PCT Filed: Aug. 4, 2022
(86) PCT No.: PCT/US2022/074512

§ 371 Date: Jan. 31, 2024

Related U.S. Application Data
(60) Provisional application No. 63/229,377, filed on Aug. 4, 2021.
(60) Provisional application No. 63/229,372, filed on Aug. 4, 2021.

(51) Int. Cl. G01N 33/68; C12N 9/12

(52) U.S. Cl. CPC G01N 33/6845; C12N 9/12; C12Y 207/11001; C12Y 207/11022; G01N 2500/02

(57) ABSTRACT

The present disclosure relates generally to biological entity-protein (e.g. protein-protein) interactions for potential therapeutic applications, and more specifically to high-throughput methods for identifying bridging molecules that induce the formation of recruited biological entity-bridging molecule-target protein complexes to effect said biological entity-protein interactions. The present disclosure also provides methods for using said bridging molecules for treatment of disorders mediated by the target protein of the recruited biological entity-molecule-target protein complex.

### FIELD

The present disclosure relates generally to biological entity-protein interactions, and more specifically to high-throughput methods for designing and identifying bridging molecules that induce the formation of biological entity-bridging molecule-protein complexes to effect said biological entity-protein interactions (e.g., protein-protein interactions (PPIs)). The present disclosure also provides methods for using said bridging molecules for therapeutic applications, such as in oncological, immunological and neurological therapies, especially wherein the oncological, immunological or neurological disorder to be treated is mediated by the protein in the biological entity-bridging molecule-protein complex.

### BACKGROUND

There has been increasing awareness of small molecule compounds behaving as so-called “molecular glues”. A molecular glue is a small molecule compound that brings together at least two proteins in very close proximity. The molecular glue binds two, or even more than two, proteins together to form a protein-molecule-protein complex. The proteins that make up the protein-molecule-protein complex do not necessarily interact with one another directly in the absence of the molecular glue, nor are they necessarily involved in related signaling pathways when separated. However, the induced proximity of the two proteins when bound to the molecular glue may have substantial downstream effects on modulating the activity of one or both proteins. For example, rapamycin binds to the proteins mTOR and FKBP12, thereby blocking the function of mTOR. The resulting loss in activity downregulates immune responses; rapamycin has been approved as an immunosuppressive drug.

In other protein-protein interactions, some molecular glues can cause protein degradation, most frequently through the proteasome pathway. The drug thalidomide binds to the E3 ligase cereblon and recruits it to degrade the transcription factor ikaros having useful anticancer effects. PROteolysis-TArgeting Chimeras (PROTACs), so called for their ability to bind E3 ubiquitin ligase with a target protein and mark the target protein for destruction by the proteasome, have been one of the more prevalent areas of research in protein-protein interactions (PPIs). Alternative approaches to promoting protein degradation with binding molecules have also been explored. For example, it has been demonstrated that a small molecule E3 binder tethered to a nucleophilic primary amine can itself be ubiquitylated via isopeptide bond formation between the primary amine and the C-termius of ubiquitin, and the resulting complex (i.e., [Ub] n-SM bound to E3 ligase) could thus be recruited to the proteasome, leading to the degradation of the target E3. J. Am. Chem. Soc. 2021, 143 (28), 10571. It has also been demonstrated that a small molecule can cause destabilization of a protein, leading to its degradation, for example through the unfolded protein response. Nat. Rev. Drug Discov. 2017, 16 (2), 101.

Biological entity-protein interactions (such as protein-protein interactions) offer a new treatment modality to modulate the activity of a target protein in a variety of ways, including binding, inhibition, activation, modification (e.g., ubiquitinylation, methylation), and/or degradation. However, as is often the case, the target protein is typically the only known starting variable and the discovery of molecular glues and PPIs, if any, is serendipitous. In the case of rapamycin and thalidomide, their molecular glue behavior and the resulting PPIs were not revealed until many decades after their biological activity was first discovered. Platforms combining high-throughput chemical synthesis and high-throughput cell-based assays have enabled the accelerated optimization of the linker between the target-protein-binding ligand and the E3 ligase ligand in PROTACs. Large libraries of linkers with varying length, polarity, and rigidity can be synthesized in a high throughput manner and the products can be directly used for screening without further purification. With such direct-to-biology approaches, the structure-activity relationships (SAR) of linkers in PROTACs are more thoroughly studied and the PROTAC design-make-test cycles could potentially be accelerated. However, despite demonstrating the feasibility and promises of high-throughput synthesis and screening, many current approaches have been limited to the optimization of the linker part, with the other two components (i.e., the target-binding ligand and the recruited-entity-binding ligand) being pre-selected and unexplored. Eur. J. Med. Chem. 2022, 236, 114317. ACS Med. Chem. Lett. 2022, 13 (7), 1182. As a result, the current optimization tools are limited by the known PPIs and could greatly benefit from the discovery of new PPIs and the corresponding bridging molecules.

Thus, there is a need for alternative methods to facilitate the discovery of biological entity-protein interactions (e.g., protein-protein interactions) for therapeutic applications, as well as methods to systematically identify and design bridging molecules (molecular glues) that mediate and promote such biological entity-protein interactions.

### BRIEF SUMMARY

The present disclosure provides methods for high-throughput engineering, including synthesis and screening, of compounds for identifying molecular glues and biological entity-protein interactions. In some embodiments, the biological entity-protein interaction is a protein-protein interaction.

In one aspect, provided herein is a method of identifying a bridging molecule that creates a recruited entity-molecule-target protein complex, comprising: selecting a target protein; selecting a template molecule known to bind to the target protein, said template molecule comprising one or more functional groups; derivatizing at least one of the one or more functional groups of the template molecule to form a library of derivatized template molecules; and screening the library of derivatized template molecules against a plurality of biological entities in the presence of the target protein. Also provided herein is a method of identifying a bridging molecule that creates a recruited entity-molecule-target protein complex, comprising: selecting a target protein; selecting a template molecule known to bind to the target protein, said template molecule comprising one or more functional groups; derivatizing at least one of the one or more functional groups of the template molecule to form a library of derivatized template molecules; and screening the template molecule and the library of derivatized template molecules against a plurality of biological entities in the presence of the target protein. In some embodiments of the present aspect, at least one of the template molecule and the derivatized template molecules forms a recruited entity-molecule-target protein complex with the target protein and at least one or more entities in the plurality of biological entities. In certain embodiments wherein at least one of the template molecule and the derivatized template molecules forms a recruited entity-molecule-target protein complex with the target protein and at least one or more entities in the plurality of biological entities, the method further comprises designating the template molecule or the derivatized template molecule in the recruited entity-molecule-target protein complex as a bridging molecule for the recruited entity and the target protein. In other embodiments of the present aspect, which may be combined with any of the preceding embodiments, the screening step comprises contacting the template molecule and the library of derivatized template molecules with the plurality of biological entities in the presence of the target protein. It will be appreciated that throughout this disclosure, references to at least one of the template molecule and the derivatized template molecules may include embodiments referring to the template molecule only, embodiments referring to at least one of the derivatized template molecules only, and embodiments referring to both the template molecule and at least one of the derivatized template molecules. Furthermore, for aspects or embodiments that do not include the template molecule or in which the template molecule is optional, references to at least one of the template molecule and the derivatized template molecules may include embodiments referring to at least one of the derivatized template molecules only.

In some embodiments, which may be combined with the preceding embodiments, the template molecule further comprises a reactive group wherein the reactive group is not derivatized during library formation and wherein the reactive group is capable of forming a covalent bond with the target protein and/or recruited entity. In other embodiments, which may be combined with the preceding embodiments, the derivatized template molecule comprises a reactive group, wherein the reactive group is capable of forming a covalent bond with the target protein and/or the recruited entity. In some embodiments, which may be combined with any of the preceding embodiments, the recruited entity is a recruited protein.

In one aspect, provided herein is a method of identifying a bridging molecule that creates a recruited protein-molecule-target protein complex, comprising: selecting a target protein; selecting a template molecule known to bind to the target protein, said template molecule comprising one or more functional groups; derivatizing at least one of the one or more functional groups of the template molecule to form a library of derivatized template molecules; and screening the library of derivatized template molecules against a plurality of proteins in the presence of the target protein. Also provided herein is a method of identifying a bridging molecule that creates a recruited protein-molecule-target protein complex, comprising: selecting a target protein; selecting a template molecule known to bind to the target protein, said template molecule comprising one or more functional groups; derivatizing at least one of the one or more functional groups of the template molecule to form a library of derivatized template molecules; and screening the template molecule and the library of derivatized template molecules against a plurality of proteins in the presence of the target protein. In some embodiments, at least one of the template molecule and the derivatized template molecules forms a recruited protein-molecule-target protein complex with the target protein and at least one or more proteins in the plurality of proteins. In certain embodiments wherein at least one of the template molecule and the derivatized template molecules forms a recruited protein-molecule-target protein complex with the target protein and at least one or more proteins in the plurality of proteins, the method further comprises designating the template molecule or the derivatized template molecule in the recruited protein-molecule-target protein complex as a bridging molecule for the recruited protein and the target protein. In certain embodiments wherein at least one of the derivatized template molecules forms a recruited protein-molecule-target protein complex with the target protein and at least one or more proteins in the plurality of proteins, the method further comprises designating the derivatized template molecule in the recruited protein-molecule-target protein complex as a bridging molecule for the recruited protein and the target protein. In yet other embodiments, the screening step comprises contacting the template molecule and the library of derivatized template molecules with the plurality of proteins in the presence of the target protein. In yet other embodiments, the screening step comprises contacting the library of derivatized template molecules with the plurality of proteins in the presence of the target protein.

In some embodiments, the screening step comprises contacting the target protein with the template molecule and the library of derivatized template molecules to form at least one molecule-target protein complex, said at least one molecule-target protein complex comprising at least one of a template molecule-target protein complex or one or more derivatized template molecule-target protein complexes; and screening the at least one molecule-target protein complex against a plurality of proteins, wherein the at least one molecule-target protein complex forms a recruited protein-molecule-target protein complex with at least one or more proteins in the plurality of proteins. In some embodiments, which may be combined with the preceding embodiments, the template molecule further comprises a reactive group wherein the reactive group is not derivatized during library formation and wherein the reactive group is capable of forming a covalent bond with the target protein. In other embodiments, which may be combined with the preceding embodiments, the template molecule further comprises a reactive group wherein the reactive group is not derivatized during library formation and wherein the reactive group is capable of forming a covalent bond with the recruited protein. In still other embodiments, which may be combined with any of the preceding embodiments, the derivatized template molecule comprises a reactive group and wherein the reactive group is capable of forming a covalent bond with the recruited protein. In still other embodiments, which may be combined with any of the preceding embodiments, the derivatized template molecule comprises a reactive group and wherein the reactive group is capable of forming a covalent bond with the target protein. In other embodiments, which may be combined with any of the foregoing, the plurality of proteins excludes the target protein.

In some embodiments of the present aspect, which may be combined with any of the preceding embodiments, the screening of the template molecule and the library of derivatized template molecules against a plurality of proteins in the presence of the target protein is performed using a high-throughput screen. In other embodiments, which may be combined with any of the preceding embodiments, the screening of the template molecule and the library of derivatized template molecules against a plurality of proteins in the presence of the target protein is performed using AlphaELISA, mass spectrometry-based screening, Western blotting, photoreactive cross-linking, photoreactive cross-linking labeling assay, inhibition assays, activation assays, degradation assays, or concentration assays. Degradation assays may include HiBiT assays. In yet other embodiments, which may be combined with any of the foregoing embodiments, the screening of the template molecule and the library of derivatized template molecules against a plurality of proteins in the presence of the target protein is performed using a cell- or lysate-based assay. In certain embodiments, the screening step is performed using a cell-based assay. In certain other embodiments, the screening step is performed using a cell lysate-based assay.

In some embodiments, the screening step comprises contacting the target protein with the library of derivatized template molecules to form at least one molecule-target protein complex, said at least one molecule-target protein complex comprising one or more derivatized template molecule-target protein complexes; and screening the at least one molecule-target protein complex against a plurality of proteins, wherein the at least one molecule-target protein complex forms a recruited protein-molecule-target protein complex with at least one or more proteins in the plurality of proteins. In some embodiments, which may be combined with the preceding embodiments, the derivatized template molecule comprises a reactive group and wherein the reactive group is capable of forming a covalent bond with the recruited protein. In still other embodiments, which may be combined with any of the preceding embodiments, the derivatized template molecule comprises a reactive group and wherein the reactive group is capable of forming a covalent bond with the target protein. In other embodiments, which may be combined with any of the foregoing, the plurality of proteins excludes the target protein.

In some embodiments of the present aspect, which may be combined with any of the preceding embodiments, the screening of the library of derivatized template molecules against a plurality of proteins in the presence of the target protein is performed using a high-throughput screen. In other embodiments, which may be combined with any of the preceding embodiments, the screening of the library of derivatized template molecules against a plurality of proteins in the presence of the target protein is performed using AlphaELISA, mass spectrometry-based screening, Western blotting, photoreactive cross-linking, photoreactive cross-linking labeling assay, inhibition assays, activation assays, degradation assays, or concentration assays. Degradation assays may include HiBiT assays. In yet other embodiments, which may be combined with any of the foregoing embodiments, the screening of the library of derivatized template molecules against a plurality of proteins in the presence of the target protein is performed using a cell- or lysate-based assay. In certain embodiments, the screening step is performed using a cell-based assay. In certain other embodiments, the screening step is performed using a cell lysate-based assay.

In some embodiments of the present aspect, which may be combined with any of the foregoing embodiments, the portion of the template molecule which binds to the target protein is known, and the derivatizing of at least one of the one or more functional groups of the template molecule to form a library of derivatized template molecules is performed on functional groups of the template molecule which do not bind to the target protein. In some embodiments, which may be further combined with any of the preceding embodiments, the one or more functional groups of the template molecule comprises one or more of an alkyl, alkenyl, alkynyl, vinyl, allyl, halide, haloalkyl, hydroxyl, alkoxy, ether, thiol, thioether, disulfide, sulfoxide, sulfone, sulfinic acid, sulfonic acid, sulfonate ester, carbonyl, carboxylic acid, anhydride, acyl halide, aryl halide, ester, aldehyde, carbonate, carbamoyl, acetal, ketal, amino, amido, carboxamido, imino, imido, nitro, nitrate, nitrite, nitroso, azido, cyano, cyanato, isocyanato, thiocyanato, isothocyanato, sulfonyl, azo, epoxide, peroxide, phenyl, phenol, aryl, heteroaryl, cycloalkyl, cycloalkenyl, cycloalkynyl, heterocycloalkyl, heterocycloalkenyl, heterocycloalkynyl, heteroalkyl, phosphate, phosphine, boronic acid, boronic acid ester, or silylether. In other embodiments, which may be further combined with any of the preceding embodiments, the step of derivatizing comprises nucleophilic substitution, nucleophilic aromatic substitution, electrophilic substitution, addition, elimination, acylation, esterification, amidation, amination oxidation, reduction, cyclization, cross-coupling or rearrangement of at least one of the one or more functional groups. In still other embodiments, which may be further combined with any of the preceding embodiments, the one or more functional groups is derivatized by chemical groups that contain chiral centers, sterically rigid moieties (e.g., phenyl ring) or sterically flexible moieties (e.g., linear alkyls), hydrogen bond donors or acceptors, polarizable or non-polarizable moieties (e.g., soft versus hard).

In some embodiments, the reactive group comprises one or more of a halo, amino, thiol, disulfide, thiirane, aziridine, alkenyl, alkynyl, ester, sulfonic acid ester, thioester, N-hydroxysucinnimidyl ester, α,β-unsaturated keto ester, acrylate, (cyano) acrylamide, epoxide, vinyl sulfone, vinyl sulfonamide, aldehyde, ketone, nitrile, sulfonyl fluoride, fluorosulfate, squaric acid derivative, propiolamide, butynamide, urea, carbamate, lactone, chlorofluoroacetamide, or beta-lactam. In some embodiments, the reactive group comprises one or more of a halo, amino, thiol, disulfide, thiirane, aziridine, alkenyl, alkynyl, ester, sulfonic acid ester, thioester, N-hydroxysucinnimidyl ester, α,β-unsaturated keto ester, acrylate, (cyano) acrylamide, epoxide, vinyl sulfone, vinyl sulfonamide, aldehyde, ketone, nitrile, sulfonyl fluoride, fluorosulfate, squaric acid derivative, propiolamide, butynamide, or beta-lactam. In other embodiments, the reactive group forms a covalent bond to one or more amino acid residue Cys, Lys, Ser, Tyr, His, Trp, Met, Asp, Glu or Thr on the target protein. In certain embodiments, the covalent bond is irreversible. In certain other embodiments, the covalent bond is reversible.

In other embodiments of the present aspect, the derivatizing step is carried out in one or more multi-well plates. In certain embodiments, the screening is carried out in the same multi-well plates of the derivatizing step. In certain other embodiments, the derivatizing step is carried out in one or more multi-well plates, some or all of the crude reaction mixture is transferred to one or more different multi-well plates for screening. In some embodiments, which may be combined with any of the preceding embodiments, the derivatizing step is performed without purification prior to the screening step.

In still other embodiments, the method of the present aspect further comprises identifying one or more proteins in the plurality of proteins that interact with the template molecule or derivatized template molecule and target protein to form a recruited protein-molecule-target protein complex. In yet other embodiments, which may be combined with any of the preceding embodiments, the method of the present aspect further comprises measuring an interaction of the recruited protein with the target protein. In certain embodiments, the interaction between the recruited protein and the target protein is modulation, binding affinity, inhibition, activation, phosphorylation, ubiquitination, acylation, inactivation, degradation, destabilization or unfolding. In certain embodiments, the interaction between the recruited protein and the target protein is modulation, binding affinity, inhibition, activation, inactivation or degradation. In still other embodiments, the binding affinity is measured by determining a member selected from the group consisting of a biological activity of the protein, a conformational state of the protein, a dissociation constant of a test ligand for the protein, an affinity constant of a test ligand for the protein, a melting temperature of the protein, and a denaturing temperature for the protein. In some embodiments, binding affinity is measured by luminescence, for example, as in a HiBiT assay.

In another aspect, provided herein is a bridging molecule as obtained according to any of the methods described herein.

In yet another aspect, provided herein is a method for modulating the activity of a target protein, comprising: contacting the target protein with a bridging molecule in the presence of a recruited entity (e.g., protein), wherein the target protein, bridging molecule and recruited protein form a recruited entity-molecule-target protein complex (e.g., recruited protein-molecule-target protein complex).

In some embodiments of the present aspect, the recruited entity (e.g., protein) induces inhibition, inactivation, activation or degradation of the target protein. In some embodiments, the contacting step is performed in vitro. In other embodiments, the contacting step is performed in vivo. In yet other embodiments, the contacting step is performed in silico. In some embodiments, the target protein is a Ras protein. In certain embodiments, the target protein is K-Ras, H-Ras, or N-Ras. In other embodiments, the target protein is a kinase. In certain embodiments, the kinase is a RAF kinase or a cyclin dependent kinase (CDK). In still other embodiments wherein the kinase is a RAF kinase, the RAF kinase is A-Raf, B-Raf, or C-Raf. In some embodiments wherein the kinase is a cyclin dependent kinase, the CDK is CDK1, CDK2, CDK3, CDK4, CDK5, CDK6, CDK7, CDK8, CDK9, CDK10, CDK11, CDK12, CDK13, CDK14, CDK15, CDK16, CDK17, CDK18, CDK19, or CDK20. In some embodiments, the inhibition, inactivation, activation or degradation of the target protein is induced by the interaction between the target protein and the recruited entity. In some embodiments wherein the degradation of the target protein is induced by the recruited entity (e.g., protein), the recruited entity is an E3 ligase. In still other embodiments, the inhibition, inactivation, activation or degradation of the target protein is induced by the bridging molecule. In still other embodiments, the bridging molecule induces the degradation of the target protein by causing the target protein to unfold, exposing a degron with the target protein, or otherwise destabilizing the target protein.

In one aspect, provided herein is a method of identifying a bridging molecule that creates a recruited entity-molecule-target protein complex, comprising: selecting a biological entity; selecting a template molecule known to bind to the biological entity, said template molecule comprising one or more functional groups; derivatizing at least one of the one or more functional groups of the template molecule to form a library of derivatized template molecules; and screening the library of derivatized template molecules against a plurality of target proteins in the presence of the biological entity. Also provided herein is a method of identifying a bridging molecule that creates a recruited entity-molecule-target protein complex, comprising: selecting a biological entity; selecting a template molecule known to bind to the biological entity, said template molecule comprising one or more functional groups; derivatizing at least one of the one or more functional groups of the template molecule to form a library of derivatized template molecules; and screening the template molecule and the library of derivatized template molecules against a plurality of target proteins in the presence of the biological entity. In some embodiments of the present aspect, at least one of the template molecule and the derivatized template molecules forms a recruited entity-molecule-target protein complex with the biological entity and at least one or more target proteins in the plurality of target proteins. In certain embodiments wherein at least one of the template molecule and the derivatized template molecules forms a recruited entity-molecule-target protein complex with the biological entity and at least one or more target proteins in the plurality of target proteins, the method further comprises designating the template molecule or the derivatized template molecule in the recruited entity-molecule-target protein complex as a bridging molecule for the target protein and the recruited entity. In other embodiments of the present aspect, which may be combined with any of the preceding embodiments, the screening step comprises contacting the template molecule and the library of derivatized template molecules with the plurality of target proteins in the presence of the biological entity. In other embodiments of the present aspect, which may be combined with any of the preceding embodiments, the screening step comprises contacting the library of derivatized template molecules with the plurality of target proteins in the presence of the biological entity.

In some embodiments, which may be combined with the preceding embodiments, the template molecule further comprises a reactive group wherein the reactive group is not derivatized during library formation and wherein the reactive group is capable of forming a covalent bond with the recruited entity and/or target protein. In other embodiments, which may be combined with the preceding embodiments, the derivatized template molecule comprises a reactive group, wherein the reactive group is capable of forming a covalent bond with the recruited entity and/or the target protein. In some embodiments, which may be combined with any of the preceding embodiments, the recruited entity is a recruited protein.

In one aspect, provided herein is a method of identifying a bridging molecule that creates a recruited protein-molecule-target protein complex, comprising: selecting a biological entity which is or comprises a protein; selecting a template molecule known to bind to the biological entity, said template molecule comprising one or more functional groups; derivatizing at least one of the one or more functional groups of the template molecule to form a library of derivatized template molecules; and screening the library of derivatized template molecules against a plurality of target proteins in the presence of the biological entity. In some embodiments, at least one of the derivatized template molecules forms a recruited protein-molecule-target protein complex with the biological entity and at least one or more target proteins in the plurality of target proteins. In certain embodiments wherein at least one of the derivatized template molecules forms a recruited protein-molecule-target protein complex with the biological entity and at least one or more target proteins in the plurality of target proteins, the method further comprises designating the derivatized template molecule in the recruited protein-molecule-target protein complex as a bridging molecule for the recruited protein and the target protein. In yet other embodiments, the screening step comprises contacting the library of derivatized template molecules with the plurality of target proteins in the presence of the biological entity. Also provided herein is a method of identifying a bridging molecule that creates a recruited protein-molecule-target protein complex, comprising: selecting a biological entity which is or comprises a protein; selecting a template molecule known to bind to the biological entity, said template molecule comprising one or more functional groups; derivatizing at least one of the one or more functional groups of the template molecule to form a library of derivatized template molecules; and screening the template molecule and the library of derivatized template molecules against a plurality of target proteins in the presence of the biological entity. In some embodiments, at least one of the template molecule and the derivatized template molecules forms a recruited protein-molecule-target protein complex with the biological entity and at least one or more target proteins in the plurality of target proteins. In certain embodiments wherein at least one of the template molecule and the derivatized template molecules forms a recruited protein-molecule-target protein complex with the biological entity and at least one or more target proteins in the plurality of target proteins, the method further comprises designating the template molecule or the derivatized template molecule in the recruited protein-molecule-target protein complex as a bridging molecule for the recruited protein and the target protein. In yet other embodiments, the screening step comprises contacting the template molecule and the library of derivatized template molecules with the plurality of target proteins in the presence of the biological entity.

In some embodiments, the screening step comprises contacting the biological entity which is or comprises a protein with the template molecule and the library of derivatized template molecules to form at least one molecule-biological entity complex, said at least one molecule-biological entity complex comprising at least one of a template molecule-biological entity complex or one or more derivatized template molecule-biological entity complexes; and screening the at least one molecule-biological entity complex against a plurality of target proteins, wherein the at least one molecule-biological entity complex forms a recruited entity-molecule-target protein complex with at least one or more target proteins in the plurality of target proteins. In some embodiments, which may be combined with the preceding embodiments, the template molecule further comprises a reactive group wherein the reactive group is not derivatized during library formation and wherein the reactive group is capable of forming a covalent bond with the biological entity. In other embodiments, which may be combined with the preceding embodiments, the template molecule further comprises a reactive group wherein the reactive group is not derivatized during library formation and wherein the reactive group is capable of forming a covalent bond with the target protein. In still other embodiments, which may be combined with any of the preceding embodiments, the derivatized template molecule comprises a reactive group and wherein the reactive group is capable of forming a covalent bond with the target protein. In still other embodiments, which may be combined with any of the preceding embodiments, the derivatized template molecule comprises a reactive group and wherein the reactive group is capable of forming a covalent bond with the biological entity. In other embodiments, which may be combined with any of the foregoing, the plurality of target proteins excludes the biological entity (when the biological entity is or comprises a protein).

In some embodiments of the present aspect, which may be combined with any of the preceding embodiments, the screening of the template molecule and the library of derivatized template molecules against a plurality of target proteins in the presence of the biological entity which is or comprises a protein is performed using a high-throughput screen. In other embodiments, which may be combined with any of the preceding embodiments, the screening of the template molecule and the library of derivatized template molecules against a plurality of target proteins in the presence of the biological entity is performed using AlphaELISA, mass spectrometry-based screening, Western blotting, photoreactive cross-linking, photoreactive cross-linking labeling assay, inhibition assays, activation assays, degradation assays, or concentration assays. Degradation assays may include HiBiT assays. In yet other embodiments, which may be combined with any of the foregoing embodiments, the screening of the template molecule and the library of derivatized template molecules against a plurality of target proteins in the presence of the biological entity is performed using a cell- or lysate-based assay. In certain embodiments, the screening step is performed using a cell-based assay. In certain other embodiments, the screening step is performed using a cell lysate-based assay.

In some embodiments, the screening step comprises contacting the biological entity with the library of derivatized template molecules to form at least one molecule-biological entity complex, said at least one molecule-biological entity complex comprising at least one of a template molecule-biological entity complex or one or more derivatized template molecule-biological entity complexes; and screening the at least one molecule-biological entity complex against a plurality of target proteins, wherein the at least one molecule-biological entity complex forms a recruited entity-molecule-target protein complex with at least one or more target proteins in the plurality of target proteins. In some embodiments, which may be combined with any of the preceding embodiments, the derivatized template molecule comprises a reactive group and wherein the reactive group is capable of forming a covalent bond with the target protein. In still other embodiments, which may be combined with any of the preceding embodiments, the derivatized template molecule comprises a reactive group and wherein the reactive group is capable of forming a covalent bond with the biological entity. In other embodiments, which may be combined with any of the foregoing, the plurality of target proteins excludes the biological entity (when the biological entity is or comprises a protein).

In some embodiments of the present aspect, which may be combined with any of the preceding embodiments, the screening of and the library of derivatized template molecules against a plurality of target proteins in the presence of the biological entity which is or comprises a protein is performed using a high-throughput screen. In other embodiments, which may be combined with any of the preceding embodiments, the screening of the library of derivatized template molecules against a plurality of target proteins in the presence of the biological entity is performed using AlphaELISA, mass spectrometry-based screening, Western blotting, photoreactive cross-linking, photoreactive cross-linking labeling assay, inhibition assays, activation assays, degradation assays, or concentration assays. Degradation assays may include HiBiT assays. In yet other embodiments, which may be combined with any of the foregoing embodiments, the screening of the library of derivatized template molecules against a plurality of target proteins in the presence of the biological entity is performed using a cell- or lysate-based assay. In certain embodiments, the screening step is performed using a cell-based assay. In certain other embodiments, the screening step is performed using a cell lysate-based assay.

In some embodiments of the present aspect, which may be combined with any of the foregoing embodiments, the portion of the template molecule which binds to the biological entity is known, and the derivatizing of at least one of the one or more functional groups of the template molecule to form a library of derivatized template molecules is performed on functional groups of the template molecule which do not bind to the biological entity. In some embodiments, which may be further combined with any of the preceding embodiments, the one or more functional groups of the template molecule comprises one or more of an alkyl, alkenyl, alkynyl, vinyl, allyl, halide, haloalkyl, hydroxyl, alkoxy, ether, thiol, thioether, disulfide, sulfoxide, sulfone, sulfinic acid, sulfonic acid, sulfonate ester, carbonyl, carboxylic acid, anhydride, acyl halide, aryl halide, ester, aldehyde, carbonate, carbamoyl, acetal, ketal, amino, amido, carboxamido, imino, imido, nitro, nitrate, nitrite, nitroso, azido, cyano, cyanato, isocyanato, thiocyanato, isothocyanato, sulfonyl, azo, epoxide, peroxide, phenyl, phenol, aryl, heteroaryl, cycloalkyl, cycloalkenyl, cycloalkynyl, heterocycloalkyl, heterocycloalkenyl, heterocycloalkynyl, heteroalkyl, phosphate, phosphine, boronic acid, boronic acid ester, or silylether. In other embodiments, which may be further combined with any of the preceding embodiments, the step of derivatizing comprises nucleophilic substitution, nucleophilic aromatic substitution, electrophilic substitution, addition, elimination, acylation, esterification, amidation, amination oxidation, reduction, cyclization, cross-coupling or rearrangement of at least one of the one or more functional groups. In still other embodiments, which may be further combined with any of the preceding embodiments, the one or more functional groups is derivatized by chemical groups that contain chiral centers, sterically rigid moieties (e.g., phenyl ring) or sterically flexible moieties (e.g., linear alkyls), hydrogen bond donors or acceptors, polarizable or non-polarizable moieties (e.g., soft versus hard).

In some embodiments, the reactive group comprises one or more of a halo, amino, thiol, disulfide, thiirane, aziridine, alkenyl, alkynyl, ester, sulfonic acid ester, thioester, N-hydroxysucinnimidyl ester, α,β-unsaturated keto ester, acrylate, (cyano) acrylamide, epoxide, vinyl sulfone, vinyl sulfonamide, aldehyde, ketone, nitrile, sulfonyl fluoride, fluorosulfate, squaric acid derivative, propiolamide, butynamide, urea, carbamate, lactone, chlorofluoroacetamide, or beta-lactam. In some embodiments, the reactive group comprises one or more of a halo, amino, thiol, disulfide, thiirane, aziridine, alkenyl, alkynyl, ester, sulfonic acid ester, thioester, N-hydroxysucinnimidyl ester, α,β-unsaturated keto ester, acrylate, (cyano) acrylamide, epoxide, vinyl sulfone, vinyl sulfonamide, aldehyde, ketone, nitrile, sulfonyl fluoride, fluorosulfate, squaric acid derivative, propiolamide, butynamide, or beta-lactam. In other embodiments, the reactive group forms a covalent bond to one or more amino acid residue Cys, Lys, Ser, Tyr, His, Trp, Met, Asp, Glu or Thr on the biological entity, when the biological entity is or comprises a protein. In certain embodiments, the covalent bond is irreversible. In certain other embodiments, the covalent bond is reversible.

In other embodiments of the present aspect, the derivatizing step is carried out in one or more multi-well plates. In certain embodiments, the screening is carried out in the same multi-well plates of the derivatizing step. In certain other embodiments, the derivatizing step is carried out in one or more multi-well plates, some or all of the crude reaction mixture is transferred to one or more different multi-well plates for screening. In some embodiments, which may be combined with any of the preceding embodiments, the derivatizing step is performed without purification prior to the screening step.

In still other embodiments, the method of the present aspect further comprises identifying one or more target proteins in the plurality of target proteins that interact with the template molecule or derivatized template molecule and biological entity to form a recruited entity-molecule-target protein complex. In yet other embodiments, which may be combined with any of the preceding embodiments, the method of the present aspect further comprises measuring an interaction of the recruited entity with the target protein. In certain embodiments, the interaction between the recruited protein and the target protein is modulation, binding affinity, inhibition, activation, phosphorylation, ubiquitination, acylation, inactivation, degradation, destablization or unfolding. In certain embodiments, the interaction between the recruited entity and the target protein is modulation, binding affinity, inhibition, activation, inactivation or degradation of the target protein. In still other embodiments, the binding affinity is measured by determining a member selected from the group consisting of a biological activity of the protein, a conformational state of the protein, a dissociation constant of a test ligand for the protein, an affinity constant of a test ligand for the protein, a melting temperature of the protein, and a denaturing temperature for the protein.

In another aspect, provided herein is a bridging molecule as obtained according to any of the methods described herein.

In yet another aspect, provided herein is a method for modulating the activity of a target protein, comprising: contacting the target protein with a bridging molecule in the presence of a recruited entity, wherein the target protein, bridging molecule and recruited entity form a recruited entity-molecule-target protein complex.

In some embodiments of the present aspect, the recruited entity induces inhibition, inactivation, activation or degradation of the target protein. In some embodiments, the contacting step is performed in vitro. In other embodiments, the contacting step is performed in vivo. In yet other embodiments, the contacting step is performed in silico. In some embodiments, the target protein is a Ras protein. In certain embodiments, the target protein is K-Ras, H-Ras, or N-Ras. In other embodiments, the target protein is a kinase. In certain embodiments, the kinase is a RAF kinase or a cyclin dependent kinase (CDK). In still other embodiments wherein the kinase is a RAF kinase, the RAF kinase is A-Raf, B-Raf, or C-Raf. In some embodiments wherein the kinase is a cyclin dependent kinase, the CDK is CDK1, CDK2, CDK3, CDK4, CDK5, CDK6, CDK7, CDK8, CDK9, CDK10, CDK11, CDK12, CDK13, CDK14, CDK15, CDK16, CDK17, CDK18, CDK19, or CDK20. In some embodiments, the inhibition, inactivation, activation or degradation of the target protein is induced by the interaction between the target protein and the recruited entity. In still other embodiments, the inhibition, inactivation, activation or degradation of the target protein is induced by the bridging molecule. In still other embodiments, the bridging molecule induces the degradation of the target protein by causing the target protein to unfold.

In some embodiments of any of the aspects described herein, the recruited entity comprises a protein. In some embodiments of any of the aspects described herein, the recruited entity is a protein. In some embodiments of any of the aspects described herein, the recruited entity is an E3 ligase. In some embodiments of any of the aspects described herein, the recruited entity is an E2 ligase. In some embodiments of any of the aspects described herein, the recruited entity is selected from the group consisting of VHL, cereblon, MDM2, an IAP, and a DCAF. In some embodiments of any of the aspects described herein, the recruited entity is selected from the group consisting of KEAP1, AHR, BIRC3, RNF4, RNF114, RNF43, RNF7, RNF130, DCAF4, DCAF1, DCAF11, XIAP, and cIAP.

Certain methods described herein are directed to identifying a bridging molecule that creates a recruited entity-molecule-target protein complex, comprising selecting a biological entity; selecting a template molecule known or predicted to bind to the biological entity, said template molecule comprising one or more functional groups; derivatizing at least one of the one or more functional groups of the template molecule to form a library of derivatized template molecules; and screening the library of derivatized template molecules against a plurality of target proteins in the presence of the biological entity. In such methods, the biological entity to which the template molecule is known or predicted to bind may be referred to herein as a recruited entity. In some embodiments, the biological entity to which the template molecule is known or predicted to bind is an E3 ligase. In some embodiments, the biological entity to which the template molecule is known or predicted to bind is an E2 ligase. In some embodiments, the biological entity to which the template molecule is known or predicted to bind is selected from the group consisting of VHL, cereblon, MDM2, an IAP, and a DCAF. In some embodiments, the biological entity to which the template molecule is known or predicted to bind is selected from the group consisting of KEAP1, AHR, BIRC3, RNF4, RNF114, RNF43, RNF7, RNF130, DCAF4, DCAF1, DCAF11, XIAP, and cIAP.

### DESCRIPTION OF THE FIGURES

The present application can be understood by reference to the following description taken in conjunction with the accompanying figures.

FIG. 1 depicts an exemplary process to identify a bridging molecule that creates a recruited protein-molecule-target protein complex, starting with selecting a target protein.

FIG. 2 depicts an exemplary process to identify a bridging molecule that creates a recruited entity-molecule-target protein complex starting with selecting a biological entity.

FIGS. 3A-3B depict an exemplary template molecule and exemplary derivatized template molecules. The template molecule contains a ligand moiety that binds to the target protein, and two functional groups for derivatization in FIG. 3A. In the derivatized template molecules in FIG. 3B, the functional groups have been replaced by various other chemical moieties (including reactive groups capable of forming covalent bonds and diversity elements).

### DETAILED DESCRIPTION

The following description sets forth exemplary methods, parameters and the like. It should be recognized, however, that such description is not intended as a limitation on the scope of the present disclosure but is instead provided as a description of exemplary embodiments. For example, certain embodiments are described with regards to identifying a bridging molecule for the formation of a recruited protein-bridging molecule-target protein complex. However, it is to be understood that other recruited biological entities may substitute for the recruited protein, for example, nucleic acids, organelles, lipid bilayers or portions thereof, and the like.

For traditional medicinal chemistry, the identification and development of a chemical compound that interacts with a single target protein generally follows a prescribed series of steps, in which a ligand for the target protein is identified and iteratively and incrementally optimized for the binding pocket of the target protein through computational and synthetic experiments. However, the same approach to build structure-activity relationships is not as readily applicable in investigating protein-protein interactions and designing molecular glues that could induce protein-protein interactions.

Due to the nature of a protein-protein interaction, any potential molecular glue may bring together two surfaces of two proteins involved. Unlike enzyme binding pockets found in single ligand-single protein binding, the combined surfaces of two proteins can constitute a significantly larger physical area to which a potential molecular glue can bind. Furthermore, because knowledge of protein partners in protein-protein interactions remains limited, there are a huge number of degrees of freedom in terms of the identities of two proteins that can form a PPI, the interfaces involved in such PPIs, the molecular glues than can induce the formation of a protein-molecule-protein complex to effect the interface and resulting PPI, the binding site(s) of the molecular glue to each protein and the binding affinity of the molecular glue to these binding sites. The interdependence of each of these degrees of freedom has presented a huge hurdle to the discovery of protein-protein interactions and development of molecular glues that promote these interactions. Even when a protein (e.g., target protein) is selected as one protein within a desired protein-protein interaction, there remains an intractable number of possible combinations of protein partners and potential molecular glue compounds to sift through.

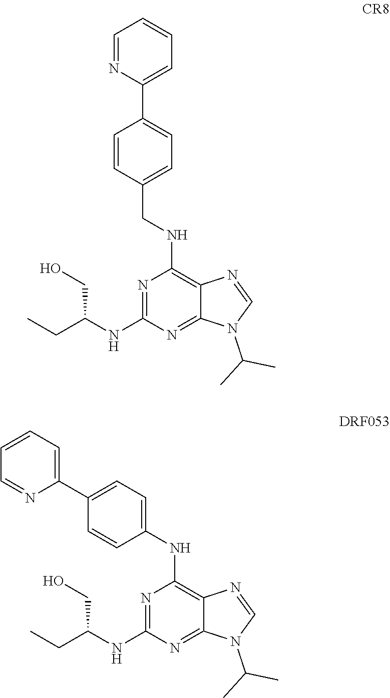 [SMILES: CC[C@H](CO)Nc1nc(NCc2ccc(-c3ccccn3)cc2)c2ncn(C(C)C)c2n1 ; CC[C@H](CO)Nc1nc(Nc2ccc(-c3ccccn3)cc2)c2ncn(C(C)C)c2n1]

Moreover, for molecular glues, even the slightest changes in the structure of the molecular glues can substantially influence binding affinity and activity of the molecular glue to either protein in a protein-molecule-protein complex, to the point of rendering the molecular glue no longer effective at being a glue. For example, one such case of a known molecular glue is the compound CR8 and its analog DRF053 above. The compound CR8 has been found to induce degradation of CDK12-cyclin K by inducing formation of a complex of CD12-cyclin K with the CUL4 adaptor protein DDB1. Biochemical and crystallographic experiments revealed that the compound CR8 acts as a molecular glue, increasing the affinity of CDK12-cyclin K for DDB1 by 500- to 1000-fold. Surprisingly, it was discovered that subtle changes to CR8 could eliminate the ability of CR8 to mediate this protein-protein interaction. The deletion of a single methylene group from CR8 was found to render the resulting analog (DRF053) ineffectual at driving formation of the ternary complex of CDK12-cyclin K and DDB1. Nat. 2020, 585 (7824), 293. In some instances, as described above, the deletion of a small and seemingly innocuous methylene group can have an unexpectedly profound and, sometimes nullifying, effect.

The current landscape of molecular glues and their protein-protein interactions remains relatively unexplored due to the added complexity of interactions between the two (or more) proteins and the molecular glue. Having two proteins interact not only with one another but also with the molecular glue adds many more degrees of freedom and uncertainty relative to the traditional-lock-and-key model associated with single protein targets and their ligands.

A recent approach involves rational design of molecules that bring two proteins together; for example, linking thalidomide derivatives to a small molecule ligand for a different protein to cause its degradation. This approach has led to several drugs in clinical development, but the resulting molecules—consisting of two separate ligands connected by what can be a long linker—are often so large that they lack good drug-like properties.

Successful, reliably effective rational design of molecular glues continues to be elusive. To date, efforts to develop molecular glues and uncover novel protein-protein interactions for therapeutic applications have remained in the realm of chance. Thus, there is a need for alternative methods to facilitate the discovery of protein-protein interactions for therapeutic applications, as well as methods to systematically identify and design bridging molecules (molecular glues) that mediate and promote such protein-protein interactions.

The present disclosure addresses this need by providing methods of identifying and designing molecular glues for inducing protein-protein (or other biological entity-protein) interactions. The methods provided herein utilize high-throughput parallel synthesis (e.g., combinatorial chemistry) and high-throughput screening to look for molecular glue behavior among libraries of small molecule compounds and to highlight potential biological entity-protein (e.g. protein-protein) interactions induced by these small molecule compounds for further investigation. The methods provided herein allow for the discovery of molecular glues and biological entity partners (e.g. protein partners) for the target protein to effect biological entity-protein interactions (e.g. protein-protein interactions), such that neither the structure of the molecular glue nor the identity of the biological entity necessarily must be known in advance.

### I. Definitions

Formation of a “complex” between recruited entity, molecule, and target protein, may be shown by demonstration of an interaction between the recruited entity and the target protein in the presence of the molecule. In some embodiments, the interaction is determined by modulation and/or binding affinity of the target protein. In various embodiments, modulation and/or binding affinity is measured by determining a member selected from the group consisting of: a biological activity of the protein, a conformational state of the protein, a dissociation constant of a test ligand for the protein, an affinity constant of a test ligand for the protein, a melting temperature of the protein, degradation of the protein, and a denaturing temperature for the protein. In various embodiments, modulation and/or binding affinity is measured by determining a member selected from the group consisting of: a biological activity of the protein, a conformational state of the protein, a dissociation constant of a test ligand for the protein, an affinity constant of a test ligand for the protein, a melting temperature of the protein, and a denaturing temperature for the protein. In various embodiments, modulation comprises inhibition, activation, inactivation, phosphorylation, ubiquitination, destabilization, unfolding, allosteric regulation of, or degradation of the target protein. In various embodiments, modulation comprises inhibition, activation, inactivation, allosteric regulation of, or degradation of the target protein.

“Recruited entity” and “recruited biological entity” are used interchangeably herein.

“Protecting group” has the meaning conventionally associated with it in organic synthesis, i.e., a group that selectively blocks one or more reactive sites in a multifunctional compound such that a chemical reaction can be carried out selectively on another unprotected reactive site, and such that the group can readily be removed after the selective reaction is complete. A variety of protecting groups are disclosed, for example, in T. H. Greene and P. G. M. Wuts, Protective Groups in Organic Synthesis, Third Edition, John Wiley & Sons, New York (1999). For example, a “hydroxy protected form” contains at least one hydroxy group protected with a hydroxy protecting group. Likewise, amines and other reactive groups may similarly be protected.

By “optional” or “optionally” is meant that the subsequently described event or circumstance may or may not occur, and that the description includes instances where the event or circumstance occurs and instances in which it does not.

The term “pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in pharmaceutical compositions is contemplated. Supplementary active ingredients can also be incorporated into the pharmaceutical compositions.

The terms “patient,” “individual,” and “subject” refer to an animal, such as a mammal, bird, or fish. In some embodiments, the patient or subject is a mammal. Mammals include, for example, mice, rats, dogs, cats, pigs, sheep, horses, cows and humans. In some embodiments, the patient, individual, or subject is a human, for example a human that has been or will be the object of treatment, observation or experiment. The compounds, compositions and methods described herein can be useful in both human therapy and veterinary applications.

The term “therapeutically effective amount” or “effective amount” refers to that amount of a compound (e.g., a bridging molecule) disclosed and/or described herein that is sufficient to effect treatment, as defined herein, when administered to a patient in need of such treatment. A therapeutically effective amount of a compound may be an amount sufficient to treat a disease responsive to modulation (e.g., inhibition) of the target protein. The therapeutically effective amount will vary depending upon, for example, the subject and disease condition being treated, the weight and age of the subject, the severity of the disease condition, the particular compound, the dosing regimen to be followed, timing of administration, the manner of administration, all of which can readily be determined by one of ordinary skill in the art. The therapeutically effective amount may be ascertained experimentally, for example by assaying blood concentration of the chemical entity, or theoretically, by calculating bioavailability.

“Treatment” (and related terms, such as “treat,” “treated,” “treating”) includes one or more of: inhibiting a disease or disorder; slowing or arresting the development of clinical symptoms of a disease or disorder; and/or relieving a disease or disorder (i.e., causing relief from or regression of clinical symptoms). The term covers both complete and partial reduction of the condition or disorder, and complete or partial reduction of clinical symptoms of a disease or disorder. Thus, compounds described and/or disclosed herein (e.g. a bridging molecule) may prevent an existing disease or disorder from worsening, assist in the management of the disease or disorder, or reduce or eliminate the disease or disorder.

It is understood that aspects and variations described herein also include “consisting” and/or “consisting essentially of” aspects and variations.

### II. Methods of High-Throughput Engineering of Molecular Glues

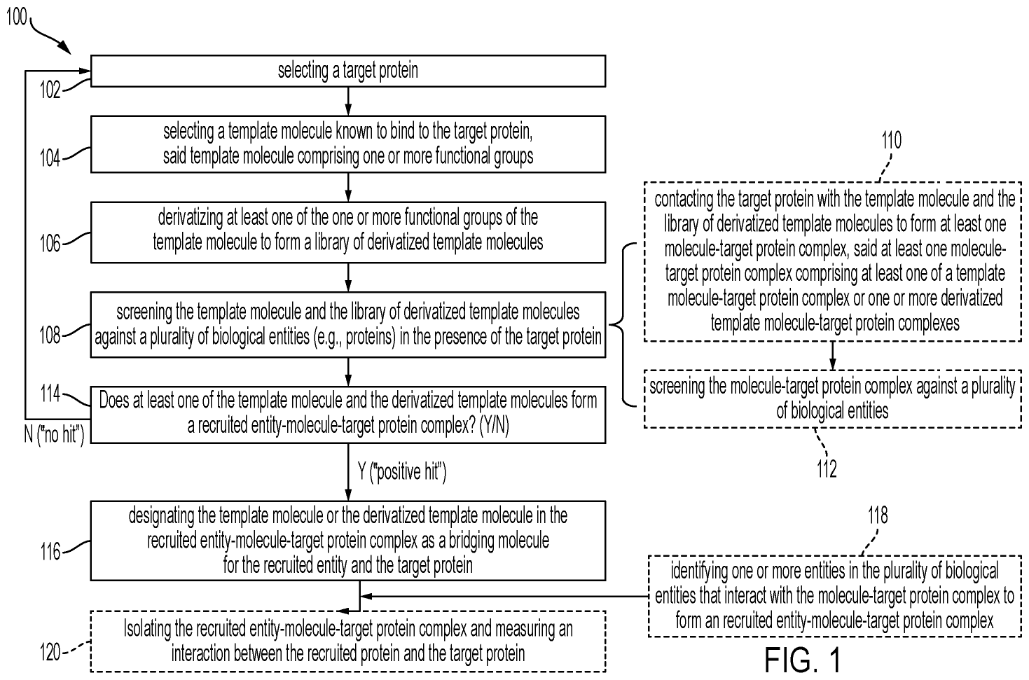

In one aspect, provided herein are methods of identifying a bridging molecule that creates a recruited entity-molecule-target protein complex (e.g. a recruited protein-molecule-target protein complex). With reference to FIG. 1, provided herein is an exemplary process 100 to identify a bridging molecule that creates a recruited protein-molecule-target protein complex. In step 102, a target protein is selected. The target protein is a protein having a known biological activity or involvement in a signaling pathway, such as an oncogenic, immunologic, or neurologic signaling pathway. The activity of the target protein is intended to be modulated in the protein-molecule-protein complex, for example, through activation, inhibition, inactivation, allosteric regulation, or even degradation of the target protein. It should be recognized that modulation of the target protein may encompass any degree of modulation of the target protein that differs from the activity of the same target protein in the absence of the bridging molecule and the recruited biological entity (e.g. recruited protein). As a non-limiting example, when the target protein is degraded in the presence of the bridging molecule and the recruited protein, the result may be complete degradation of the target protein (100%), or partial degradation of the target protein (e.g., greater than 0% but less than 100%). In some embodiments, the degree of modulation of the target protein is sufficient to elicit a therapeutic effect in a subject.

In step 104, a template molecule known to bind to the target protein is selected. The template molecule contains one or more functional groups, which may be further modified. Again, with reference to FIG. 1, at least one of the one or more functional groups present on the template molecule are derivatized to form a library of derivatized template molecules in step 106. Once the library of derivatized template molecules has been generated, the library of derivatized template molecules, and optionally also the template molecule, is screened against a plurality of proteins in the presence of the target protein in step 108. In some variations of step 108, at least one of the template molecule, if present, and derivatized template molecules forms at least one three-(or greater than three-) component recruited protein(s)-molecule-target protein complex. If no protein-molecule-protein complexes are observed or otherwise determined to form, that is, if no “positive hits” are found in the screen, steps 102 through 108 may be repeated until a positive hit is found. The recruited protein molecule-target protein complex formed in step 108 may be formed between the template molecule, the target protein, and one or more of the proteins in the plurality of proteins or between a derivatized template molecule, the target protein, and one or more of the proteins in the plurality of proteins.

In some variations, the screening step of step 108 may encompass combining or contacting the plurality of proteins, the template molecule, if present, and library of derivatized template molecules and target protein in any sequential order, such as combining the template molecule, if present, and library derivatized template molecules with the plurality of proteins first and then adding the target protein or, alternatively combining the target protein and plurality of proteins first and then adding the template molecule and library derivatized template molecules. In certain variations, the screening step of step 108 may involve first contacting the template molecule, if present, and the derivatized template molecules with the target protein to form at least one binary molecule-target protein complex, as shown in step 110, and then further screening the molecule-target protein complex against the plurality of proteins in step 112. In other variations of step 108, the plurality of proteins, the template molecule and library of derivatized template molecules and target protein may be combined or contacted simultaneously.

In other variations of steps 108, 110, and 112, it should be further recognized that the template molecule, if present, and the library of derivatized templates molecules may be screened in the presence of the target protein against a plurality of biological entities, not limited solely to proteins, which could exhibit modulation of the target protein. For example, in some embodiments, the screening step is performed using a cell-based or cell lysate-based assay, wherein the plurality of biological entities that may be recruited to form a ternary complex with the (derivatized) template molecule and the target protein may include but are not limited to nucleic acids (such as ribonucleic acids, deoxyribonucleic acids, etc.), proteins and enzymes (which may be intracellular, extracellular, and/or transmembrane), organelles, or lipid bilayer membranes or fragments thereof. In some variations, the template molecule, if present, and the library of derivatized templates molecules may be screened in the presence of the target protein against a plurality of biological entities, wherein at least one template molecule or derivatized template molecule forms at least one recruited entity-molecule-target protein complex. It should be recognized that, for any embodiments describing a recruited protein as provided herein, variations of those embodiments as they would apply to any other recruited biological entities, such as recruited nucleic acids, recruited membranes, etc., are also contemplated. For example, in embodiments wherein the template molecule and the library of derivatized templates molecules may be screened in the presence of the target protein against a plurality of biological entities, the template molecule, if present, and the library of derivatized templates molecules, the target protein, and the plurality of biological entities may be contacted or combined in any sequential order or simultaneously.

With further reference to FIG. 1, in step 108, and optionally, in step 112, the screening step involves a high-throughput screen against a plurality of biological entities in which formation of a ternary complex composed of a (derivatized) template molecule, the target protein and a biological entity can occur. Once the template molecule, if present, and the library of derivatized templates molecules, the biological entity, and the plurality of target proteins are contacted or combined as part of the screening step, the subsequent formation of a ternary complex may be determined using techniques known in the art, for example, using AlphaLISA (amplified luminescent proximity homogeneous assay), in step 114.

A protein within the plurality of proteins screened and determined to exhibit an interaction with the (derivatized) template molecule and target protein, that is, determined to be a “positive hit”, is denoted as a “recruited protein”. Similarly, any biological entity within the plurality of biological entities screened and determined to exhibit an interaction with the (derivatized) template molecule and target protein, that is, determined to be a “positive hit”, is denoted as a “recruited entity” and the template molecule or derivatized template molecule mediating the interaction between the recruited protein (or entity) and target protein is designated as a “bridging molecule” in step 116.

With reference to FIG. 1, in optional step 118, any proteins from the plurality of proteins (or other biological entities) that interact with the (derivatized) template molecule and target protein complex to form a recruited protein (entity)-molecule-target protein complex may be identified. In some variations of step 118 wherein a ternary complex is formed from the template or derivatized template molecule, the target protein, and a biological entity, the identity of the recruited entity may be similarly identified.

Again, with reference to FIG. 1, in optional step 120, the recruited protein (entity)-molecule-target protein complex may be isolated and the interaction between the recruited protein (entity) and the bridging molecule-target protein complex measured.

In one aspect, provided herein is a method of identifying a bridging molecule that creates a recruited entity-molecule-target protein complex, comprising: selecting a target protein; selecting a template molecule known to bind to the target protein, said template molecule comprising one or more functional groups; derivatizing at least one of the one or more functional groups of the template molecule to form a library of derivatized template molecules; and screening the library of derivatized template molecules against a plurality of biological entities (e.g., nucleic acids, proteins, enzymes, organelles, and/or membranes) in the presence of the target protein. In some embodiments, at least one of the derivatized template molecules forms a recruited entity-molecule-target protein complex with the target protein and at least one or more biological entities in the plurality of biological entities. In certain embodiments wherein at least one of the derivatized template molecules forms a recruited entity-molecule-target protein complex with the target protein and at least one or more biological entities in the plurality of biological entities, the method further comprises designating the derivatized template molecule in the recruited entity-molecule-target protein complex as a bridging molecule for the recruited entity and the target protein. Also provided herein is a method of identifying a bridging molecule that creates a recruited entity-molecule-target protein complex, comprising: selecting a target protein; selecting a template molecule known to bind to the target protein, said template molecule comprising one or more functional groups; derivatizing at least one of the one or more functional groups of the template molecule to form a library of derivatized template molecules; and screening the template molecule and the library of derivatized template molecules against a plurality of biological entities (e.g., nucleic acids, proteins, enzymes, organelles, and/or membranes) in the presence of the target protein. In some embodiments, at least one of the template molecule and the derivatized template molecules forms a recruited entity-molecule-target protein complex with the target protein and at least one or more biological entities in the plurality of biological entities. In certain embodiments wherein at least one of the template molecule and the derivatized template molecules forms a recruited entity-molecule-target protein complex with the target protein and at least one or more biological entities in the plurality of biological entities, the method further comprises designating the template molecule or the derivatized template molecule in the recruited entity-molecule-target protein complex as a bridging molecule for the recruited entity and the target protein.

In another aspect, provided herein is a method of identifying a bridging molecule that creates a recruited protein-molecule-target protein complex, comprising: selecting a target protein; selecting a template molecule known to bind to the target protein, said template molecule comprising one or more functional groups; derivatizing at least one of the one or more functional groups of the template molecule to form a library of derivatized template molecules; and screening the library of derivatized template molecules against a plurality of proteins. Also provided herein is a method of identifying a bridging molecule that creates a recruited protein-molecule-target protein complex, comprising: selecting a target protein; selecting a template molecule known to bind to the target protein, said template molecule comprising one or more functional groups; derivatizing at least one of the one or more functional groups of the template molecule to form a library of derivatized template molecules; and screening the template molecule and the library of derivatized template molecules against a plurality of proteins. In some embodiments, at least one of the template molecule and the derivatized template molecules forms a recruited protein-molecule-target protein complex with the target protein and at least one or more proteins in the plurality of proteins. In certain embodiments wherein at least one of the template molecule and the derivatized template molecules forms a recruited protein-molecule-target protein complex with the target protein and at least one or more proteins in the plurality of proteins, the method further comprises designating the template molecule or the derivatized template molecule in the recruited protein-molecule-target protein complex as a bridging molecule for the recruited protein and the target protein.

In yet another aspect, provided herein is a method of identifying a bridging molecule that creates a recruited protein-molecule-target protein complex, comprising: selecting a target protein; selecting a template molecule known to bind to the target protein, said template molecule comprising one or more functional groups; derivatizing at least one of the one or more functional groups of the template molecule to form a library of derivatized template molecules; screening the library of derivatized template molecules against a plurality of proteins in the presence of the target protein, wherein at least one of the derivatized template molecules forms a recruited protein-molecule-target protein complex with the target protein and at least one or more proteins in the plurality of proteins; and designating the derivatized template molecule in the recruited protein-molecule-target protein complex as a bridging molecule for the recruited protein and the target protein. Also provided herein is a method of identifying a bridging molecule that creates a recruited protein-molecule-target protein complex, comprising: selecting a target protein; selecting a template molecule known to bind to the target protein, said template molecule comprising one or more functional groups; derivatizing at least one of the one or more functional groups of the template molecule to form a library of derivatized template molecules; screening the template molecule and the library of derivatized template molecules against a plurality of proteins in the presence of the target protein, wherein at least one of the template molecule and the derivatized template molecules forms a recruited protein-molecule-target protein complex with the target protein and at least one or more proteins in the plurality of proteins; and designating the template molecule or the derivatized template molecule in the recruited protein-molecule-target protein complex as a bridging molecule for the recruited protein and the target protein.

In still yet another aspect, provided herein is a method of identifying a bridging molecule that creates a recruited entity-molecule-target protein complex, comprising: selecting a target protein; selecting a template molecule known to bind to the target protein, said template molecule comprising one or more functional groups; derivatizing at least one of the one or more functional groups of the template molecule to form a library of derivatized template molecules; contacting the target protein with the library of derivatized template molecules to form at least one molecule-target protein complex, said at least one molecule-target protein complex comprising one or more derivatized template molecule-target protein complexes; screening the molecule-target protein complex against a plurality of biological entities; identifying one or more biological entities in the plurality of biological entities that interact with the molecule-target protein complex to form a recruited entity-molecule-target protein complex; and designating the derivatized template molecule in the recruited entity-molecule-target protein complex as a bridging molecule for the recruited entity and the target protein. Also provided herein is a method of identifying a bridging molecule that creates a recruited entity-molecule-target protein complex, comprising: selecting a target protein; selecting a template molecule known to bind to the target protein, said template molecule comprising one or more functional groups; derivatizing at least one of the one or more functional groups of the template molecule to form a library of derivatized template molecules; contacting the target protein with the template molecule and the library of derivatized template molecules to form at least one molecule-target protein complex, said at least one molecule-target protein complex comprising at least one of a template molecule-target protein complex or one or more derivatized template molecule-target protein complexes; screening the molecule-target protein complex against a plurality of biological entities; identifying one or more biological entities in the plurality of biological entities that interact with the molecule-target protein complex to form a recruited entity-molecule-target protein complex; and designating the template molecule or the derivatized template molecule in the recruited entity-molecule-target protein complex as a bridging molecule for the recruited entity and the target protein.

In still yet another aspect, provided herein is a method of identifying a bridging molecule that creates a recruited protein-molecule-target protein complex, comprising: selecting a target protein; selecting a template molecule known to bind to the target protein, said template molecule comprising one or more functional groups; derivatizing at least one of the one or more functional groups of the template molecule to form a library of derivatized template molecules; contacting the target protein with the library of derivatized template molecules to form at least one molecule-target protein complex, said at least one molecule-target protein complex comprising one or more derivatized template molecule-target protein complexes; screening the molecule-target protein complex against a plurality of proteins; identifying one or more proteins in the plurality of proteins that interact with the molecule-target protein complex to form a recruited protein-molecule-target protein complex; and designating the derivatized template molecule in the recruited protein-molecule-target protein complex as a bridging molecule for the recruited protein and the target protein. Also provided herein is a method of identifying a bridging molecule that creates a recruited protein-molecule-target protein complex, comprising: selecting a target protein; selecting a template molecule known to bind to the target protein, said template molecule comprising one or more functional groups; derivatizing at least one of the one or more functional groups of the template molecule to form a library of derivatized template molecules; contacting the target protein with the template molecule and the library of derivatized template molecules to form at least one molecule-target protein complex, said at least one molecule-target protein complex comprising at least one of a template molecule-target protein complex or one or more derivatized template molecule-target protein complexes; screening the molecule-target protein complex against a plurality of proteins; identifying one or more proteins in the plurality of proteins that interact with the molecule-target protein complex to form a recruited protein-molecule-target protein complex; and designating the template molecule or the derivatized template molecule in the recruited protein-molecule-target protein complex as a bridging molecule for the recruited protein and the target protein.

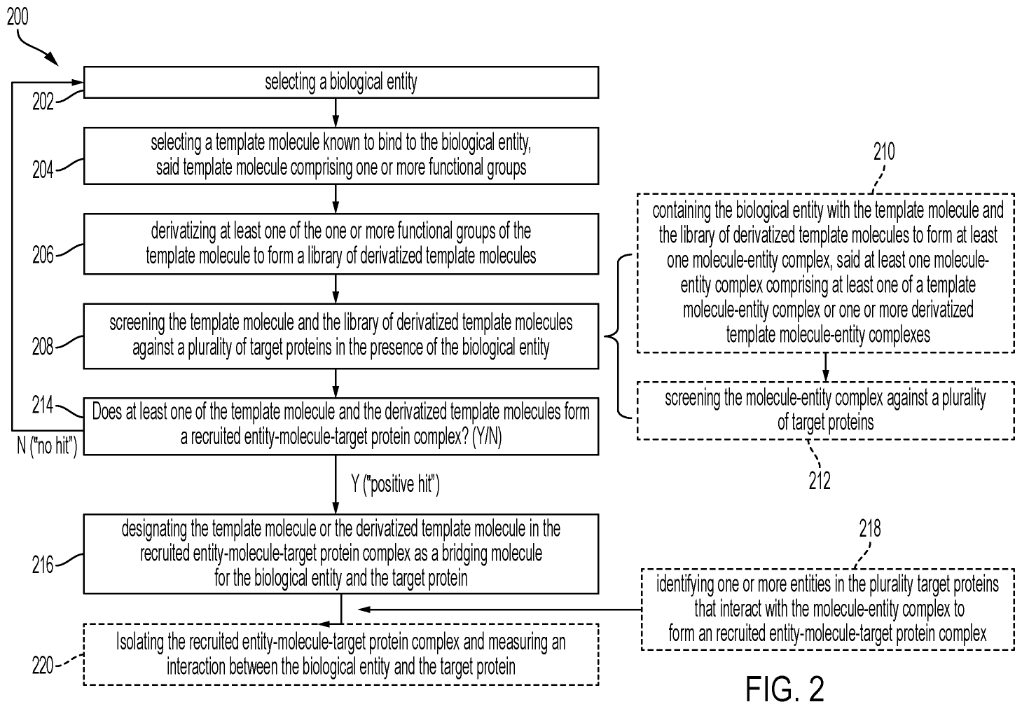

In another aspect, provided herein are methods of identifying a bridging molecule that creates a recruited entity-molecule-target protein complex (e.g., a recruited protein-molecule-target protein complex). With reference to FIG. 2, provided herein is an exemplary process 200 to identify a bridging molecule that creates a recruited entity-molecule-target protein complex. In step 202, a biological entity is selected. The biological entity has a known or anticipated function (e.g., protease or ligase) and is desired to act upon and modulate the activity or concentration of a target protein yet to be identified by the methods herein. The activity of the target protein is intended to be modulated in the biological entity-molecule-target protein complex, for example, through activation, inhibition, inactivation, phosphorylation, ubiquitination, allosteric regulation, or even degradation of the target protein. In some embodiments, the activity of the target protein is intended to be modulated by inducing the degradation of the target protein by causing the target protein to unfold. In some embodiments, the activity of the target protein is intended to be modulated by inducing destabilization of the target protein. It should be recognized that modulation of the target protein may encompass any degree of modulation of the target protein that differs from the activity of the same target protein in the absence of the bridging molecule and the recruited biological entity (e.g. recruited protein). As a non-limiting example, when the target protein is degraded in the presence of the bridging molecule and the recruited entity (e.g., protein), the result may be complete degradation of the target protein (100%), or partial degradation of the target protein (e.g., greater than 0% but less than 100%). In some embodiments, the degree of modulation of the target protein is sufficient to elicit a therapeutic effect in a subject.

In step 204, a template molecule known to bind to the biological entity is selected. The template molecule contains one or more functional groups, which may be further modified. Again, with reference to FIG. 2, at least one of the one or more functional groups present on the template molecule are derivatized to form a library of derivatized template molecules in step 206. Once the library of derivatized template molecules has been generated, the library of derivatized template molecules, and optionally also the template molecule, is screened against a plurality of target proteins in the presence of the biological entity in step 208. In some variations of step 208, at least one of the template molecule, if present, and derivatized template molecules forms at least one three-(or greater than three-) component recruited entity(s)-molecule-target protein complex. If no entity-molecule-protein complexes are observed or otherwise determined to form, that is, if no “positive hits” are found in the screen, steps 202 through 208 may be repeated until a positive hit is found. The recruited entity-molecule-target protein complex formed in step 208 may be formed between the template molecule, the biological entity, and one or more of the target proteins in the plurality of target proteins or between a derivatized template molecule, the biological entity, and one or more of the target proteins in the plurality of target proteins.

In some variations, the screening step of step 208 may encompass combining or contacting the plurality of target proteins, the template molecule, if present, and library of derivatized template molecules and biological entity in any sequential order, such as combining the template molecule and library derivatized template molecules with the plurality of target proteins first and then adding the biological entity or, alternatively combining the biological entity and plurality of target proteins first and then adding the template molecule and library derivatized template molecules. In certain variations, the screening step of step 208 may involve first contacting the template molecule, if present, and the derivatized template molecules with the biological entity to form at least one binary molecule-biological entity complex, as shown in step 210, and then further screening the molecule-biological entity complex against the plurality of target proteins in step 212. In other variations of step 208, the plurality of target proteins, the template molecule, if present, and library of derivatized template molecules and biological entity may be combined or contacted simultaneously.

In other variations of steps 208, 210, and 212, it should be further recognized that the biological entity may comprise one or more of: nucleic acids (such as ribonucleic acids, deoxyribonucleic acids, etc.), proteins and enzymes (which may be intracellular, extracellular, and/or transmembrane), organelles, or lipid bilayer membranes or fragments thereof. In some embodiments, the screening step is performed using a cell-based or cell lysate-based assay. In some variations, the template molecule and the library of derivatized templates molecules may be screened in the presence of the biological entity against a plurality of target proteins, wherein at least one template molecule or derivatized template molecule forms at least one recruited entity-molecule-target protein complex. It should be recognized that, for any embodiments describing a recruited protein as provided herein, variations of those embodiments as they would apply to any other recruited biological entities, such as recruited nucleic acids, recruited membranes, etc., are also contemplated. For example, in embodiments wherein the library of derivatized templates molecules may be screened in the presence of the biological entity against a plurality of target proteins, the library of derivatized templates molecules, the biological entity, and the plurality of target proteins may be contacted or combined in any sequential order or simultaneously. For another example, in embodiments wherein the template molecule and the library of derivatized templates molecules may be screened in the presence of the biological entity against a plurality of target proteins, the template molecule and the library of derivatized templates molecules, the biological entity, and the plurality of target proteins may be contacted or combined in any sequential order or simultaneously.

With further reference to FIG. 2, in step 208, and optionally, in step 212, the screening step involves a high-throughput screen against a plurality of target proteins in which formation of a ternary complex composed of a (derivatized) template molecule, the biological entity and a target protein can occur. Once the template molecule, if present, and the library of derivatized templates molecules, the biological entity, and the plurality of target proteins are contacted or combined as part of the screening step, the subsequent formation of a ternary complex may be determined using techniques known in the art, for example, using AlphaLISA (amplified luminescent proximity homogeneous assay), in step 214.

When a target protein within the plurality of target proteins is screened and determined to exhibit an interaction with the (derivatized) template molecule and biological entity, that is, determined to be a “positive hit”, then the biological entity is denoted as a “recruited entity” for that target protein. Similarly, when a target protein within the plurality of target proteins is screened and determined to exhibit an interaction with the (derivatized) template molecule and biological entity that is or comprises a protein, that is, determined to be a “positive hit”, then the biological entity is denoted as a “recruited protein” and the template molecule or derivatized template molecule mediating the interaction between the recruited protein (or entity) and target protein is designated as a “bridging molecule” in step 216.

With reference to FIG. 2, in optional step 218, any target proteins from the plurality of target proteins that interact with the (derivatized) template molecule and biological entity (e.g. protein) complex to form a recruited entity (protein)-molecule-target protein complex may be identified. In some variations of step 218 wherein a ternary complex is formed from the template or derivatized template molecule, the target protein, and a biological entity, the identity of the target protein may be similarly identified.

Again, with reference to FIG. 2, in optional step 220, the recruited entity (protein)-molecule-target protein complex may be isolated and the interaction between the target protein and the bridging molecule-entity (protein) complex measured.

In yet another aspect, provided herein is a method of identifying a bridging molecule that creates a recruited entity-molecule-target protein complex, comprising: selecting a biological entity (e.g., nucleic acids, proteins, enzymes, organelles, and/or membranes); selecting a template molecule known to bind to the biological entity, said template molecule comprising one or more functional groups; derivatizing at least one of the one or more functional groups of the template molecule to form a library of derivatized template molecules; and screening the library of derivatized template molecules against a plurality of target proteins in the presence of the biological entity. Also provided herein is a method of identifying a bridging molecule that creates a recruited entity-molecule-target protein complex, comprising: selecting a biological entity (e.g., nucleic acids, proteins, enzymes, organelles, and/or membranes); selecting a template molecule known to bind to the biological entity, said template molecule comprising one or more functional groups; derivatizing at least one of the one or more functional groups of the template molecule to form a library of derivatized template molecules; and screening the template molecule and the library of derivatized template molecules against a plurality of target proteins in the presence of the biological entity. In some embodiments, at least one of the template molecule and the derivatized template molecules forms a recruited entity-molecule-target protein complex with the biological entity and at least one or more target proteins in the plurality of target proteins. In certain embodiments wherein at least one of the template molecule and the derivatized template molecules forms a recruited entity-molecule-target protein complex with the biological entity and at least one or more target proteins in the plurality of target proteins, the method further comprises designating the template molecule or the derivatized template molecule in the recruited entity-molecule-target protein complex as a bridging molecule for the target protein and the biological entity.

In yet another aspect, provided herein is a method of identifying a bridging molecule that creates a recruited protein-molecule-target protein complex, comprising: selecting a biological entity that is or comprises a protein; selecting a template molecule known to bind to the biological entity, said template molecule comprising one or more functional groups; derivatizing at least one of the one or more functional groups of the template molecule to form a library of derivatized template molecules; and screening the library of derivatized template molecules against a plurality of target proteins. Also provided herein is a method of identifying a bridging molecule that creates a recruited protein-molecule-target protein complex, comprising: selecting a biological entity that is or comprises a protein; selecting a template molecule known to bind to the biological entity, said template molecule comprising one or more functional groups; derivatizing at least one of the one or more functional groups of the template molecule to form a library of derivatized template molecules; and screening the template molecule and the library of derivatized template molecules against a plurality of target proteins. In some embodiments, at least one of the template molecule and the derivatized template molecules forms a recruited protein-molecule-target protein complex with the biological entity and at least one or more target proteins in the plurality of target proteins. In certain embodiments wherein at least one of the template molecule and the derivatized template molecules forms a recruited protein-molecule-target protein complex with the biological entity and at least one or more target proteins in the plurality of target proteins, the method further comprises designating the template molecule or the derivatized template molecule in the recruited protein-molecule-target protein complex as a bridging molecule for the recruited protein and the target protein.

In yet another aspect, provided herein is a method of identifying a bridging molecule that creates a recruited protein-molecule-target protein complex, comprising: selecting a biological entity that is or comprises a protein; selecting a template molecule known to bind to the biological entity, said template molecule comprising one or more functional groups; derivatizing at least one of the one or more functional groups of the template molecule to form a library of derivatized template molecules; screening the library of derivatized template molecules against a plurality of target proteins in the presence of the biological entity, wherein at least one of the derivatized template molecules forms a recruited protein-molecule-target protein complex with the biological entity and at least one or more target proteins in the plurality of target proteins; and designating the derivatized template molecule in the recruited protein-molecule-target protein complex as a bridging molecule for the recruited protein and the target protein. Also provided herein is a method of identifying a bridging molecule that creates a recruited protein-molecule-target protein complex, comprising: selecting a biological entity that is or comprises a protein; selecting a template molecule known to bind to the biological entity, said template molecule comprising one or more functional groups; derivatizing at least one of the one or more functional groups of the template molecule to form a library of derivatized template molecules; screening the template molecule and the library of derivatized template molecules against a plurality of target proteins in the presence of the biological entity, wherein at least one of the template molecule and the derivatized template molecules forms a recruited protein-molecule-target protein complex with the biological entity and at least one or more target proteins in the plurality of target proteins; and designating the template molecule or the derivatized template molecule in the recruited protein-molecule-target protein complex as a bridging molecule for the recruited protein and the target protein.

In still yet another aspect, provided herein is a method of identifying a bridging molecule that creates a recruited entity-molecule-target protein complex, comprising: selecting a biological entity; selecting a template molecule known to bind to the biological entity, said template molecule comprising one or more functional groups; derivatizing at least one of the one or more functional groups of the template molecule to form a library of derivatized template molecules; contacting the biological entity with the library of derivatized template molecules to form at least one molecule-biological entity complex, said at least one molecule-biological entity complex comprising one or more derivatized template molecule-biological entity complexes; screening the molecule-biological entity complex against a plurality of target proteins; identifying one or more target proteins in the plurality of target proteins that interact with the molecule-biological entity complex to form a recruited entity-molecule-target protein complex; and designating the derivatized template molecule in the recruited entity-molecule-target protein complex as a bridging molecule for the recruited entity and the target protein. Also provided herein is a method of identifying a bridging molecule that creates a recruited entity-molecule-target protein complex, comprising: selecting a biological entity; selecting a template molecule known to bind to the biological entity, said template molecule comprising one or more functional groups; derivatizing at least one of the one or more functional groups of the template molecule to form a library of derivatized template molecules; contacting the biological entity with the template molecule and the library of derivatized template molecules to form at least one molecule-biological entity complex, said at least one molecule-biological entity complex comprising at least one of a template molecule-biological entity complex or one or more derivatized template molecule-biological entity complexes; screening the molecule-biological entity complex against a plurality of target proteins; identifying one or more target proteins in the plurality of target proteins that interact with the molecule-biological entity complex to form a recruited entity-molecule-target protein complex; and designating the template molecule or the derivatized template molecule in the recruited entity-molecule-target protein complex as a bridging molecule for the recruited entity and the target protein.

In still yet another aspect, provided herein is a method of identifying a bridging molecule that creates a recruited entity-molecule-target protein complex (e.g., recruited protein-molecule-target protein complex), comprising: selecting a biological entity that is or comprises a protein; selecting a template molecule known to bind to the biological entity, said template molecule comprising one or more functional groups; derivatizing at least one of the one or more functional groups of the template molecule to form a library of derivatized template molecules; contacting the biological entity with the library of derivatized template molecules to form at least one molecule-biological entity complex, said at least one molecule-biological entity complex comprising one or more derivatized template molecule-biological entity complexes; screening the molecule-biological entity complex against a plurality of target proteins; identifying one or more target proteins in the plurality of target proteins that interact with the molecule-biological entity complex to form a recruited entity-molecule-target protein complex (e.g., recruited protein-molecule-target protein complex); and designating the derivatized template molecule in the recruited entity-molecule-target protein complex (e.g., recruited protein-molecule-target protein complex) as a bridging molecule for the recruited entity (e.g., protein) and the target protein. Also provided herein is a method of identifying a bridging molecule that creates a recruited entity-molecule-target protein complex (e.g., recruited protein-molecule-target protein complex), comprising: selecting a biological entity that is or comprises a protein; selecting a template molecule known to bind to the biological entity, said template molecule comprising one or more functional groups; derivatizing at least one of the one or more functional groups of the template molecule to form a library of derivatized template molecules; contacting the biological entity with the template molecule and the library of derivatized template molecules to form at least one molecule-biological entity complex, said at least one molecule-biological entity complex comprising at least one of a template molecule-biological entity complex or one or more derivatized template molecule-biological entity complexes; screening the molecule-biological entity complex against a plurality of target proteins; identifying one or more target proteins in the plurality of target proteins that interact with the molecule-biological entity complex to form a recruited entity-molecule-target protein complex (e.g., recruited protein-molecule-target protein complex); and designating the template molecule or the derivatized template molecule in the recruited entity-molecule-target protein complex (e.g., recruited protein-molecule-target protein complex) as a bridging molecule for the recruited entity (e.g., protein) and the target protein.

### Target Protein(s)

The methods described herein may be employed to identify or discover a recruited entity capable of modulating a target protein. As provided herein, the method of identifying a bridging molecule that creates a recruited entity-molecule-target protein complex comprises selecting a target protein. The target protein as provided herein may be a protein, the activity or concentration of which is desired to be modulated by interaction with a biological entity (referred to herein as a “biological entity partner” or “recruited entity”) in a biological entity-protein interaction. In some embodiments, the biological entity is a second protein (referred to herein as a “protein partner” or “recruited protein”) in a protein-protein interaction.

For a given target protein, a particular type of modulation of its activity may be desired. For example, in circumstances in which a target protein is overexpressed or otherwise activated, the desired modulation of the target protein may be inhibition, inactivation or degradation.

Suitable target proteins may include but are not limited to kinases such as RAF kinases and CDKs and proteins such as Ras proteins. In some embodiments, the target protein is a protein involved in an oncogenic signaling pathway. In some embodiments, the target protein is a RAF kinase. In some embodiments, the target protein is a cyclin dependent kinase (CDK). In other embodiments, the target protein is a Ras protein.

### Biological Entities

The methods described herein may be employed to identify or discover a target protein capable of being modulated by a known recruited entity, such as an E3 ligase (e.g, cereblon). Provided herein is a method of identifying a bridging molecule that creates a recruited entity-molecule-target protein complex comprising selecting a biological entity. The biological entity may also be referred to as a recruited entity. In such methods, the template molecule is known or predicted to bind to the biological (e.g., recruited) entity. A library of derivatized template molecules may be prepared based on such template molecule, and this library may be employed to discover one or more target proteins capable of being modulated by the biological (e.g., recruited entity). The biological (e.g., recruited) entity as provided herein may comprise a nucleic acid (such as ribonucleic acids, deoxyribonucleic acids, etc.), protein or enzyme (which may be intracellular, extracellular, and/or transmembrane), organelle, or lipid bilayer membrane or fragment thereof, and the like. In some embodiments, the biological entity is a protein. In some embodiments the biological entity comprises a protein. In some embodiments, the biological entity which comprises a protein binds to the bridging molecule and/or the target protein through the protein portion of the biological entity.

The biological entity may modulate the target protein in the presence of the appropriate bridging molecule, for example, by modulation of the activity or concentration of the target protein, including, e.g, activation, inhibition, inactivation, or degradation. In some embodiments, formation of the recruited entity-bridging molecule-target protein results in degradation of the target protein. In some embodiments where the biological entity is or comprises a protein, formation of the recruited protein-bridging molecule-target protein results in degradation of the target protein. In some embodiments, the recruited protein has a known enzymatic function, such as proteolysis or ubiquitination. In some embodiments, the recruited protein has a known enzymatic function, such as proteolysis.

Non-limiting examples of suitable biological entities include proteins involved in degradation of target proteins, including for example, adaptor proteins, autophagy proteins, and E2 and E3 ligases. Non-limiting examples of E3 ligases are described in Zheng, N., and Shabek, N. (2017). Ubiquitin ligases: structure, function, and regulation. Annu. Rev. Biochem. 86, 129-157; and George A J, Hoffiz Y C, Charles A J, Zhu Y and Mabb A M (2018) A Comprehensive Atlas of E3 Ubiquitin Ligase Mutations in Neurological Disorders. Front. Genet. 9:29. doi: 10.3389/fgene.2018.0002. In some embodiments of any of the aspects described herein, the recruited entity is selected from the group consisting of VHL, cereblon, MDM2, an IAP, and a DCAF. In some embodiments of any of the aspects described herein, the recruited entity is selected from the group consisting of KEAP1, AHR, BIRC3, RNF4, RNF114, RNF43, RNF7, RNF130, DCAF4, DCAF1, DCAF11, XIAP, and cIAP.

### Template Molecule(s) and Derivatization Thereof

For the methods described herein that may be employed to identify or discover a recruited entity capable of modulating a target protein, following selection of a target protein, the methods of the present disclosure may further comprise selecting a template molecule known to bind to the target protein. The template molecule serves as the starting structure for derivatization to establish a library of compounds to be screened as potential molecular glues.

As a starting skeleton for preparing a library of diverse compounds to be evaluated as potential molecular glues, the template molecule is known or expected or predicted to have some affinity for the target protein and has at least one functional group that can be derivatized. Binding affinity can be measured by a variety of methods known in the art, such as by determining a biological activity of the protein, a conformational state of the protein, a dissociation constant of a test ligand for the protein, an affinity constant of a test ligand for the protein, a melting temperature of the protein, or a denaturing temperature for the protein. Binding can also be measured by biophysical methods known in the art, such as nuclear magnetic resonance (NMR), isothermal titration calorimetry (ITC), surface plasmon resonance (SPR), microscale thermophoresis (MST), or X-ray crystallography. In some embodiments, the template molecule has a dissociation constant (K_{d}) for the target protein of less than or equal to 10 mM, less than or equal to 10 μM, or less than or equal to 100 nM. However, other characteristics of the template molecule may be favorable in designing the library of derivatized template molecules, including but not limited to low molecular weight, structural flexibility or rigidity, low lipophilicity, good permeability and stability, chirality, etc. In some embodiments, the template molecule is derived from or is an analog of a natural product. In some embodiments, the template molecule is an amino acid. In other embodiments, the template molecule comprises a synthetic small molecule. A “template molecule known to bind to the target protein” includes template molecules known or expected or predicted to have some affinity for the target protein. In some embodiments, the template molecule is known to have some affinity for the target protein, for example, as reported in literature, through experimental testing, or as predicted by homology modeling or computational docking. In some embodiments, the template molecule is predicted to have some affinity for the target protein based on quantitative or qualitative assessment.

In some embodiments, the template molecule comprises one or more chiral centers.

In some embodiments, the template molecule comprises one or more functional groups, two or more functional groups, or three or more functional groups. In certain embodiments, the template molecule comprises one or more functional groups. The one or more functional groups present on the template molecule may include but are not limited to functional groups such as alkyl, alkenyl, alkynyl, vinyl, allyl, halide, haloalkyl, hydroxyl, alkoxy, ether, thiol, thioether, disulfide, sulfoxide, sulfone, sulfinic acid, sulfonic acid, sulfonate ester, carbonyl, carboxylic acid, anhydride, acyl halide, aryl halide, ester, aldehyde, carbonate, carbamoyl, acetal, ketal, amino, amido, carboxamido, imino, imido, nitro, nitrate, nitrite, nitroso, azido, cyano, cyanato, isocyanato, thiocyanato, isothocyanato, sulfonyl, azo, epoxide, peroxide, phenyl, phenol, aryl, heteroaryl, cycloalkyl, cycloalkenyl, cycloalkynyl, heterocycloalkyl, heterocycloalkenyl, heterocycloalkynyl, heteroalkyl, phosphate, phosphine, boronic acid, boronic acid ester, or silylether.

In some embodiments wherein the template molecule comprises one or more functional groups, the method further comprises derivatizing at least one of the one or more functional groups of the template molecule to form a library of derivatized template molecules. In some embodiments, the derivatizing step comprises reacting at least one of the one or more functional groups of the template molecule. In other embodiments wherein the template molecule comprises two or more functional groups, the derivatizing step comprises reacting at least two of the two or more functional groups. It should be recognized that the derivatizing step may comprise reacting as few as one functional group on the template molecule up to as many as all of the functional groups present on the template molecule. In certain embodiments, the derivatizing step may comprise reacting two or more functional groups with each other.

In yet other embodiments, the derivatizing step may comprise any number of reaction types depending upon the functional groups present on the template molecule. Suitable derivatization reactions may include but are not limited to nucleophilic substitution, esterification, amidation, amination oxidation, reduction, cyclization, cross-coupling and rearrangement.

As described above, the template molecule is in some embodiments known or in other embodiments predicted to bind to the target protein. However, the nature of the binding interaction between the target protein and the template molecule may or may not be known or may or may not be predicted. If the specific ligand moiety or functional group that binds to the target protein is known, the derivatization approach of the template molecule may be designed and undertaken to avoid modification of that portion of the template molecule. In still other embodiments wherein the portion of the template molecule which binds to the target protein is known or is predicted, the derivatizing step is performed on one or more functional groups of the template molecule which do not bind to the target protein.

The template molecule may further comprise a reactive group capable of forming a covalent bond with the target protein. In still other embodiments, the template molecule further comprises a reactive group wherein the reactive group is not derivatized during library formation and wherein the reactive group is capable of forming a covalent bond with the target protein. In other embodiments, the template molecule comprises a reactive group capable of forming a covalent bond with a biological entity, such as a protein, to be recruited into a ternary complex with the target protein and the template molecule or the derivatized template molecule. In some variations, the covalent bond with the target protein or recruited entity (e.g. recruited protein) may be irreversible or reversible. In some embodiments, the covalent bond is irreversible. In other embodiments, the covalent bond is reversible.

In some embodiments, the reactive group comprises one or more of a halo, amino, thiol, disulfide, thiirane, aziridine, alkenyl, alkynyl, ester, sulfonic acid ester, thioester, N-hydroxysucinnimidyl ester, α,β-unsaturated keto ester, acrylate, (cyano) acrylamide, epoxide, vinyl sulfone, vinyl sulfonamide, aldehyde, ketone, nitrile, sulfonyl fluoride, fluorosulfate, squaric acid derivative, propiolamide, butynamide, urea, carbamate, lactone, chlorofluoroacetamide, or beta-lactam. In some embodiments, the reactive group comprises one or more of a halo, amino, thiol, disulfide, thiirane, aziridine, alkenyl, alkynyl, ester, sulfonic acid ester, thioester, N-hydroxysucinnimidyl ester, α,β-unsaturated keto ester, acrylate, (cyano) acrylamide, epoxide, vinyl sulfone, vinyl sulfonamide, aldehyde, ketone, nitrile, sulfonyl fluoride, fluorosulfate, squaric acid derivative, propiolamide, butynamide, or beta-lactam. In some embodiments, the reactive group forms a covalent bond to one or more amino acid residue Cys, Lys, Ser, Tyr, His, Trp, Met, Asp, Glu or Thr on the target protein. In other embodiments, the reactive group forms a covalent bond to one or more amino acid residue Cys, Lys, Ser, Tyr, His, Trp, Met, Asp, Glu or Thr on the recruited protein.

For the methods described herein that may be employed to identify or discover a target protein capable of being modulated by a known biological (e.g., recruited) entity, following selection of a biological entity (e.g., a biological entity which is a protein), the methods of the present disclosure may further comprise selecting a template molecule known to bind to the biological entity. The template molecule serves as the starting structure for derivatization to establish a library of compounds to be screened as potential molecular glues.

As a starting skeleton for preparing a library of diverse compounds to be evaluated as potential molecular glues, the template molecule is known or expected or predicted to have some affinity for the biological entity and has at least one functional group that can be derivatized. Binding affinity can be measured by a variety of methods known in the art, such as by determining a biological activity of the protein, a conformational state of the protein, a dissociation constant of a test ligand for the protein, an affinity constant of a test ligand for the protein, a melting temperature of the protein, or a denaturing temperature for the protein. Binding can also be measured by biophysical methods known in the art, such as nuclear magnetic resonance (NMR), isothermal titration calorimetry (ITC), surface plasmon resonance (SPR), microscale thermophoresis (MST), or X-ray crystallography. In some embodiments, the template molecule has a dissociation constant (K_{d}) for the biological entity of less than or equal to 10 mM, less than or equal to 10 μM, or less than or equal to 100 nM. However, other characteristics of the template molecule may be favorable in designing the library of derivatized template molecules, including but not limited to low molecular weight, structural flexibility or rigidity, low lipophilicity, good permeability and stability, chirality, etc. In some embodiments, the template molecule is derived from or is an analog of a natural product. In some embodiments, the template molecule is an amino acid. In other embodiments, the template molecule comprises a synthetic small molecule. A “template molecule known to bind to the biological entity” includes template molecules known or expected or predicted to have some affinity for the biological entity. In some embodiments, the template molecule is known to have some affinity for the biological entity, for example, as reported in literature, through experimental testing, or as predicted by homology modeling or computational docking. In some embodiments, the template molecule is predicted to have some affinity for the target protein based on quantitative or qualitative assessment.

In some embodiments, the template molecule comprises one or more chiral centers.

In some embodiments, the template molecule comprises one or more functional groups, two or more functional groups, or three or more functional groups. In certain embodiments, the template molecule comprises one or more functional groups. The one or more functional groups present on the template molecule may include but are not limited to functional groups such as alkyl, alkenyl, alkynyl, vinyl, allyl, halide, haloalkyl, hydroxyl, alkoxy, ether, thiol, thioether, disulfide, sulfoxide, sulfone, sulfinic acid, sulfonic acid, sulfonate ester, carbonyl, carboxylic acid, anhydride, acyl halide, aryl halide, ester, aldehyde, carbonate, carbamoyl, acetal, ketal, amino, amido, carboxamido, imino, imido, nitro, nitrate, nitrite, nitroso, azido, cyano, cyanato, isocyanato, thiocyanato, isothocyanato, sulfonyl, azo, epoxide, peroxide, phenyl, phenol, aryl, heteroaryl, cycloalkyl, cycloalkenyl, cycloalkynyl, heterocycloalkyl, heterocycloalkenyl, heterocycloalkynyl, heteroalkyl, phosphate, phosphine, boronic acid, boronic acid ester, or silylether.

In some embodiments wherein the template molecule comprises one or more functional groups, the method further comprises derivatizing at least one of the one or more functional groups of the template molecule to form a library of derivatized template molecules. In some embodiments, the derivatizing step comprises reacting at least one of the one or more functional groups of the template molecule. In other embodiments wherein the template molecule comprises two or more functional groups, the derivatizing step comprises reacting at least two of the two or more functional groups. It should be recognized that the derivatizing step may comprise reacting as few as one functional group on the template molecule up to as many as all of the functional groups present on the template molecule. In certain embodiments, the derivatizing step may comprise reacting two or more functional groups with each other.

In yet other embodiments, the derivatizing step may comprise any number of reaction types depending upon the functional groups present on the template molecule. Suitable derivatization reactions may include but are not limited to nucleophilic substitution, esterification, amidation, amination oxidation, reduction, cyclization, cross-coupling and rearrangement.

As described above, the template molecule is in some embodiments known and in other embodiments predicted to bind to the biological entity. However, the nature of the binding interaction between the biological entity and the template molecule may or may not be known or may or may not be predicted. If the specific ligand moiety or functional group that binds to the biological entity is known, the derivatization approach of the template molecule may be designed and undertaken to avoid modification of that portion of the template molecule. In still other embodiments wherein the portion of the template molecule which binds to the biological entity is known or is predicted, the derivatizing step is performed on one or more functional groups of the template molecule which do not bind to the biological entity.

The template molecule may further comprise a reactive group capable of forming a covalent bond with the biological entity. In still other embodiments, the template molecule further comprises a reactive group wherein the reactive group is not derivatized during library formation and wherein the reactive group is capable of forming a covalent bond with the biological entity. In other embodiments, the template molecule comprises a reactive group capable of forming a covalent bond with a target protein. In some variations, the covalent bond with the recruited entity (e.g. recruited protein) or target protein may be irreversible or reversible. In some embodiments, the covalent bond is irreversible. In other embodiments, the covalent bond is reversible.

In some embodiments, the reactive group comprises one or more of a halo, amino, thiol, disulfide, thiirane, aziridine, alkenyl, alkynyl, ester, sulfonic acid ester, thioester, N-hydroxysucinnimidyl ester, α,β-unsaturated keto ester, acrylate, (cyano) acrylamide, epoxide, vinyl sulfone, vinyl sulfonamide, aldehyde, ketone, nitrile, sulfonyl fluoride, fluorosulfate, squaric acid derivative, propiolamide, butynamide, urea, carbamate, lactone, chlorofluoroacetamide, or beta-lactam. In some embodiments, the reactive group comprises one or more of a halo, amino, thiol, disulfide, thiirane, aziridine, alkenyl, alkynyl, ester, sulfonic acid ester, thioester, N-hydroxysucinnimidyl ester, α,β-unsaturated keto ester, acrylate, (cyano) acrylamide, epoxide, vinyl sulfone, vinyl sulfonamide, aldehyde, ketone, nitrile, sulfonyl fluoride, fluorosulfate, squaric acid derivative, propiolamide, butynamide, or beta-lactam. In some embodiments, the reactive group forms a covalent bond to one or more amino acid residue Cys, Lys, Ser, Tyr, His, Trp, Met, Asp, Glu or Thr on the target protein. In other embodiments, the reactive group forms a covalent bond to one or more amino acid residue Cys, Lys, Ser, Tyr, His, Trp, Met, Asp, Glu or Thr on the recruited protein.

### Derivatized Template Molecule(s), Molecule-Target Protein Complexes, Molecule-Biological Entity Complexes, and Libraries Thereof

The derivatization of the template molecule as detailed above results in the production of a library of derivatized template molecules.

In some embodiments, the library of derivatized template molecules comprises at least about 5 derivatized template molecules, at least about 10 derivatized template molecules, at least about 100 derivatized template molecules, at least about 1,000 derivatized template molecules, at least about 5,000 derivatized template molecules, or at least about 10,000 derivatized template molecules. In other embodiments, the library of derivatized template molecules comprises less than or equal to about 50,000 derivatized template molecules, less than or equal to about 25,000 derivatized template molecules, less than or equal to about 20,000 derivatized template molecules, less than or equal to about 10,000 derivatized template molecules, less than or equal to about 5,000 derivatized template molecules, less than or equal to about 2,000 derivatized template molecules, less than or equal to about 1,000 derivatized template molecules, or less than or equal to about 100 derivatized template molecules. In certain embodiments, the library of derivatized template molecules comprises between about 5 derivatized template molecules and about 20,000 derivatized template molecules.

In some embodiments, the library of derivatized template molecules may be designed in order to optimize breadth and coverage of the chemical space of the derivatized template molecules. In some embodiments, the library of derivatized template molecules is a diversity-oriented library. Prior to the actual process of derivatization, for example, computational analysis of divergent synthesis protocols may be conducted to assess the diversity of the proposed library of derivatized molecules and to guide the choice of chemical reactions employed in high-throughput parallel synthesis. In some embodiments, the derivatizing step comprises divergent synthesis. In some embodiments, the derivatizing step comprises one or more combinatorial synthetic techniques, such as immobilization methods (e.g., solid-phase synthesis), labeling or coding techniques (e.g., fluorescent labeling, isotopic labeling), and pooling. In some embodiments, the derivatizing step is carried out in one or more multi-well plates.

For the methods described herein that may be employed to identify or discover a recruited entity capable of modulating a target protein, the derivatizing step as detailed above may introduce chemical moieties onto the template molecule that provide a specific, intended purpose in the resulting derivatized template molecule, such as a reactive group capable of forming a covalent bond with either the target protein or the (yet-to-be) recruited entity (e.g. recruited protein).

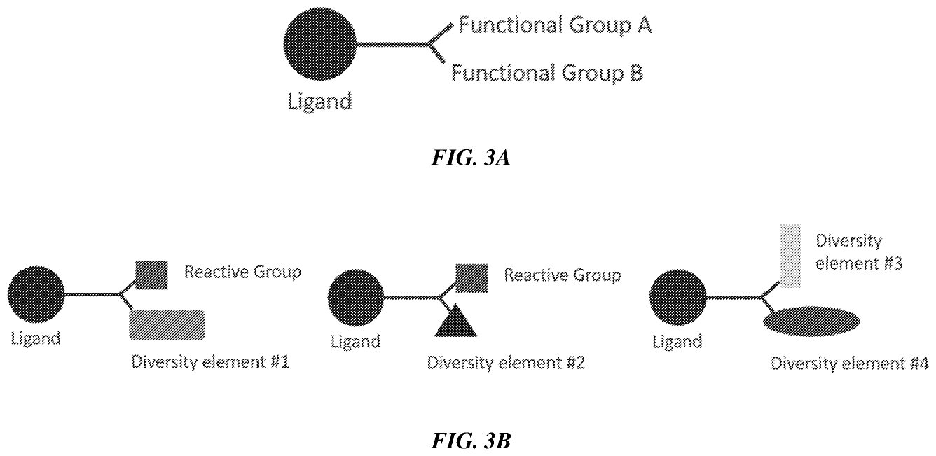

In still other embodiments, the derivatizing step described above may introduce chemical moieties, or diversity elements, intended to increase structural diversity of the library of compounds to be screened. In some embodiments, the derivatized template molecule comprises one or more diversity elements. Suitable diversity elements may include but are not limited to chemical groups that contain chiral centers, sterically rigid moieties (e.g., phenyl ring) or sterically flexible moieties (e.g., linear alkyls), hydrogen bond donors or acceptors, polarizable or non-polarizable moieties (e.g., soft versus hard). FIGS. 3A-3B can be an illustration of the derivatization of functional group on a template molecule having a ligand moiety capable of binding to the target protein and two functional groups (FIG. 3A) to produce three distinct derivatized template molecules with different reactive groups and diversity elements (FIG. 3B). In some embodiments, the one or more functional groups of the template molecule is derivatized by a diversity element. In certain embodiments, the one or more functional groups of the template molecule is derivatized by chemical groups that contain chiral centers, sterically rigid moieties (e.g., phenyl ring) or sterically flexible moieties (e.g., linear alkyls), hydrogen bond donors or acceptors, polarizable or non-polarizable moieties (e.g., soft versus hard). In other embodiments, the one or more functional groups of the template molecule is derivatized by one or more reactive groups capable of forming a covalent bond with a protein. In other embodiments, the one or more functional groups of the template molecule is derivatized by one or more reactive groups capable of forming a covalent bond with a biological entity, such as a nucleic acid, a protein, an enzyme, an organelle or a membrane. It should be noted that that the Figure is illustrative only and meant to be non-limiting. For example, the ligand could have only one functional group, or more than two. Similarly, any reactive group on the ligand does not need to be in close proximity to the functional group(s).

In other embodiments, the derivatized template molecule comprises one or more reactive groups capable of forming a covalent bond with a protein. In some embodiments, the derivatized template molecule further comprises a reactive group, wherein the reactive group is capable of forming a covalent bond with the target protein. The reactive group capable of forming a covalent bond with the target protein may have been present in the template molecule or may have been introduced in the derivatizing step. In other embodiments, the derivatized template molecule comprises a reactive group, wherein the reactive group is capable of forming a covalent bond with the recruited protein. In certain embodiments, a reactive group is capable of forming a covalent bond with the recruited protein. In some embodiments, the one or more functional groups of the template molecule is derivatized by a reactive group capable of forming a covalent bond with the target protein or the recruited protein. In still other embodiments, the derivatized template molecules comprises at least two reactive groups capable of forming a covalent bond with the target protein and/or the recruited protein. In some embodiments, the one or more functional groups of the template molecule is derivatized by halo, thiol, disulfide, thiirane, aziridine, alkenyl, alkynyl, ester, sulfonic acid ester, thioester, N-hydroxysucinnimidyl ester, α,β-unsaturated keto ester, acrylate, (cyano) acrylamide, epoxide, vinyl sulfone, vinyl sulfonamide, aldehyde, ketone, nitrile, sulfonyl fluoride, fluorosulfate, squaric acid derivative, propiolamide, butynamide, urea, carbamate, lactone, chlorofluoroacetamide or beta-lactam. In some embodiments, the one or more functional groups of the template molecule is derivatized by halo, thiol, disulfide, thiirane, aziridine, alkenyl, alkynyl, ester, sulfonic acid ester, thioester, N-hydroxysucinnimidyl ester, α,β-unsaturated keto ester, acrylate, (cyano) acrylamide, epoxide, vinyl sulfone, vinyl sulfonamide, aldehyde, ketone, nitrile, sulfonyl fluoride, fluorosulfate, squaric acid derivative, propiolamide, butynamide, or beta-lactam. In some variations, the covalent bond with the protein may be irreversible or reversible. In some embodiments, the covalent bond is irreversible. In other embodiments, the covalent bond is reversible.

In still other embodiments wherein the portion of the template molecule that binds to the target protein is known, the derivatized template molecule comprises the same portion of the template molecule that binds to the target protein. It should be recognized, however, that in some embodiments, the portion of the template molecule that binds to the target protein may not be known and may be altered or removed by the derivatization process, resulting in reduced or null binding to the target protein by the resulting derivatized template molecule.

The high-throughput derivatization process of the present methods provides a library of hundreds, or even thousands, of individually unique analogs of the template molecule to be screened for evidence of biological activity towards the target protein, particularly the ability of the derivatized molecule(s) to recruit a second, yet to be recruited protein (or other biological entity), forming a complex with the target protein. Each derivatized template molecule is structurally unique with respect to other derivatized template molecules within the same library of compounds. As such, it should be recognized that each derivatized template molecule independently comprises different combinations of chemical moieties.

In some embodiments, a derivatized template molecule may comprise one or more diversity elements, one or more reactive groups capable of forming a covalent bond with the target protein or with the recruited protein, or a portion of the template molecule that binds to the target protein, or any combinations thereof. In certain embodiments, a derivatized template molecule comprises at least one diversity element, and optionally one or more reactive groups capable of forming a covalent bond with the target protein or with the recruited entity (e.g. recruited protein), or a portion of the template molecule that binds to the target protein.

For the methods described herein that may be employed to identify or discover a target protein capable of being modulated by a known biological (e.g., recruited) entity, the derivatizing step as detailed above may introduce chemical moieties onto the template molecule that provide a specific, intended purpose in the resulting derivatized template molecule, such as a reactive group capable of forming a covalent bond with either the recruited entity (e.g. recruited protein) or the (yet-to-be identified) target protein.

In still other embodiments, the derivatizing step described above may introduce chemical moieties, or diversity elements, intended to increase structural diversity of the library of compounds to be screened. In some embodiments, the derivatized template molecule comprises one or more diversity elements. Suitable diversity elements may include but are not limited to chemical groups that contain chiral centers, sterically rigid moieties (e.g., phenyl ring) or sterically flexible moieties (e.g., linear alkyls), hydrogen bond donors or acceptors, polarizable or non-polarizable moieties (e.g., soft versus hard). FIGS. 3A-3B can be an illustration of the derivatization of functional group on a template molecule having a ligand moiety capable of binding to the biological entity and two functional groups (FIG. 3A) to produce three distinct derivatized template molecules with different reactive groups and diversity elements (FIG. 3B). In some embodiments, the one or more functional groups of the template molecule is derivatized by a diversity element. In certain embodiments, the one or more functional groups of the template molecule is derivatized by chemical groups that contain chiral centers, sterically rigid moieties (e.g., phenyl ring) or sterically flexible moieties (e.g., linear alkyls), hydrogen bond donors or acceptors, polarizable or non-polarizable moieties (e.g., soft versus hard). In other embodiments, the one or more functional groups of the template molecule is derivatized by one or more reactive groups capable of forming a covalent bond with a protein. In other embodiments, the one or more functional groups of the template molecule is derivatized by one or more reactive groups capable of forming a covalent bond with a biological entity, such as a nucleic acid, a protein, an enzyme, an organelle or a membrane.

In other embodiments, the derivatized template molecule comprises one or more reactive groups capable of forming a covalent bond with a protein. In some embodiments, the derivatized template molecule further comprises a reactive group, wherein the reactive group is capable of forming a covalent bond with the biological entity. The reactive group capable of forming a covalent bond with the biological entity may have been present in the template molecule or may have been introduced in the derivatizing step. In other embodiments, the derivatized template molecule comprises a reactive group, wherein the reactive group is capable of forming a covalent bond with the target protein. In certain embodiments, a reactive group is capable of forming a covalent bond with the target protein. In some embodiments, the one or more functional groups of the template molecule is derivatized by a reactive group capable of forming a covalent bond with the target protein or the recruited entity (e.g., recruited protein). In still other embodiments, the derivatized template molecules comprises at least two reactive groups capable of forming a covalent bond with the target protein and/or the recruited entity (e.g. recruited protein). In some embodiments, the one or more functional groups of the template molecule is derivatized by halo, thiol, disulfide, thiirane, aziridine, alkenyl, alkynyl, ester, sulfonic acid ester, thioester, N-hydroxysucinnimidyl ester, α,β-unsaturated keto ester, acrylate, (cyano) acrylamide, epoxide, vinyl sulfone, vinyl sulfonamide, aldehyde, ketone, nitrile, sulfonyl fluoride, fluorosulfate, squaric acid derivative, propiolamide, butynamide, urea, carbamate, lactone, chlorofluoroacetamide or beta-lactam. In some embodiments, the one or more functional groups of the template molecule is derivatized by halo, thiol, disulfide, thiirane, aziridine, alkenyl, alkynyl, ester, sulfonic acid ester, thioester, N-hydroxysucinnimidyl ester, α,β-unsaturated keto ester, acrylate, (cyano) acrylamide, epoxide, vinyl sulfone, vinyl sulfonamide, aldehyde, ketone, nitrile, sulfonyl fluoride, fluorosulfate, squaric acid derivative, propiolamide, butynamide, or beta-lactam. In some variations, the covalent bond with the protein may be irreversible or reversible. In some embodiments, the covalent bond is irreversible. In other embodiments, the covalent bond is reversible.

In still other embodiments wherein the portion of the template molecule that binds to the biological entity is known, the derivatized template molecule comprises the same portion of the template molecule that binds to the biological entity. It should be recognized, however, that in some embodiments, the portion of the template molecule that binds to the biological entity may not be known and may be altered or removed by the derivatization process, resulting in reduced or null binding to the biological entity by the resulting derivatized template molecule.

The high-throughput derivatization process of the present methods provides a library of hundreds, or even thousands, of individually unique analogs of the template molecule to be screened for evidence of biological activity towards the biological entity, particularly the ability of the derivatized molecule(s) to recruit the biological entity to a target protein, forming a complex with the target protein. Each derivatized template molecule is structurally unique with respect to other derivatized template molecules within the same library of compounds. As such, it should be recognized that each derivatized template molecule independently comprises different combinations of chemical moieties.

In some embodiments, a derivatized template molecule may comprise one or more diversity elements, one or more reactive groups capable of forming a covalent bond with the target protein or with the biological entity (e.g., protein), or a portion of the template molecule that binds to the biological entity, or any combinations thereof. In certain embodiments, a derivatized template molecule comprises at least one diversity element, and optionally one or more reactive groups capable of forming a covalent bond with the biological entity (e.g. protein) or with the target protein, or a portion of the template molecule that binds to the biological entity (e.g., protein).

### Plurality of Proteins (or Other Biological Entities) for Screening

For the methods described herein that may be employed to identify or discover a recruited entity capable of modulating a target protein, the methods of the present disclosure may further comprise screening each derivatized template molecule, and optionally also the template molecule, against a plurality of proteins in the presence of the target protein in order to determine whether any of the proteins within the plurality of proteins binds with the derivatized template molecule and target protein to form a recruited protein-molecule-target protein complex. The plurality of proteins against which the molecule-target protein complexes are screened constitute a sample population of proteins which could be identified as potential partners of the target protein for protein-protein interactions and may form a ternary complex with the molecule-target protein complex. A “positive hit” in the screen would indicate potential formation of a recruited protein-molecule-target protein complex. The recruited protein-molecule-target protein complex as used herein is referred to interchangeably with the terms “protein-molecule-protein complex”.

In some embodiments, the plurality of proteins comprises a complete cellular proteome. In certain embodiments, the screening comprises a cell-based or lysate-based screen. In some embodiments, the plurality of proteins includes the target protein. In other embodiments, the plurality of proteins excludes the target protein. The screening as provided herein allows for the discovery of protein interaction partners for the target protein, such that the identity of the protein partner need not be known prior to the screen and can be identified in subsequent work-up as a “recruited protein”.

As described above, it should be recognized that the derivatized template molecules and target protein may form ternary complexes with biological entities other than proteins that may be present in cells or cell lysates, including but not limited to nucleic acids, organelles, and/or lipid membranes. Such complexes may be referred to as “recruited entity-molecule-target protein complexes”, wherein the biological entity which engages the target protein and the derivatized template molecule is referred to as the “recruited entity”. It should be recognized that the term “ternary complex” may also be used to refer to a recruited protein-molecule-target protein complex, or protein-molecule-protein complex, or a recruited entity-molecule-target protein complexes, if only one entity or protein is recruited by the molecule and target protein. It should be further recognized that more than one biological entity or protein may be recruited into interactions with the derivatized template molecule and target protein, which could result in a greater than three-way interaction between the two or more recruited entities, the derivatized template molecule and the target protein.

In some embodiments, the screening of the library of derivatized template molecules against a plurality of biological entities (e.g., a plurality of proteins) in the presence of the target protein comprises contacting the library of derivatized template molecules with the plurality of biological entities (e.g., the plurality of proteins) in the presence of the target protein. In some embodiments, the screening of the template molecule and library of derivatized template molecules against a plurality of biological entities (e.g., a plurality of proteins) in the presence of the target protein comprises contacting the template molecule and library of derivatized template molecules with the plurality of biological entities (e.g., the plurality of proteins) in the presence of the target protein.

In some embodiments, the screening of the library of derivatized template molecules against the plurality of proteins (or other biological entities) in the presence of the target protein is carried out in one step, wherein the library of derivatized template molecules are combined with the target protein and the plurality of proteins to be screened simultaneously. In other embodiments, the screening of the library of derivatized template molecules may be carried out in at least two sequential steps. For example, in some embodiments, the library of derivatized template molecules is first contacted with the target protein to form a library of molecule-target protein complexes. In some embodiments, the screening of the template molecule and the library of derivatized template molecules against the plurality of proteins (or other biological entities) in the presence of the target protein is carried out in one step, wherein the template molecule and the library of derivatized template molecules are combined with the target protein and the plurality of proteins to be screened simultaneously. In other embodiments, the screening of the template molecule and the library of derivatized template molecules may be carried out in at least two sequential steps. For example, in some embodiments, the library of derivatized template molecules, and optionally also the template molecule, is first contacted with the target protein to form a library of molecule-target protein complexes. As noted above, in circumstances wherein the binding portion of the template molecule is not known, the derivatizing step could potentially reduce or eliminate the binding affinity of the derivatized template molecules for the target protein. In some embodiments, at least one molecule-target protein complex comprises at least one template molecule-target protein complex.

The template molecule may also be contacted with the target protein to form a template molecule-target protein complex, which may then be employed as a control sample in evaluating the binding behavior of the derivatized molecules. In other embodiments, at least one molecule-target protein complex comprises at least one template molecule-target protein complex.

Suitable screening assays may include but are not limited to assays such as, Western blotting, AlphaLISA, mass spectrometry-based screening, or screens that indicate modulation (such as inhibition, activation, or inactivation) or disappearance (e.g., degradation) of the target protein. In some embodiments, the step of screening comprises a high-throughput screen, wherein the library of molecule-target protein complexes is screened against the plurality of proteins in parallel. In some embodiments, the screening is carried out in one or more multi-well plates. In other embodiments wherein the derivatizing step is carried out in one or more multi-well plates, the screening is carried out in the same multi-well plates of the derivatizing step. In certain embodiments wherein the derivatizing step is carried out in one or more multi-well plates, the screening is carried out in the same multi-well plates of the derivatizing step without the need for purification of the library members prior to the screening step. In certain embodiments wherein the derivatizing step is carried out in one or more multi-well plates, some or all of the crude reaction mixture is transferred to one or more different multi-well plates for screening.

In some embodiments, the screening step comprises screening for formation of a recruited entity-molecule-target protein complex. In some embodiments, the screening step comprises screening for formation of a recruited protein-molecule-target protein complex. The formation of a recruited protein-molecule-target protein complex may be observed using distance- or proximity-dependent assays, such as time-resolved fluorescence resonance energy transfer (TR-FRET), AlphaLISA amplified luminescent proximity homogeneous assays, photoreactive cross-linking labeling assays, etc. In still other embodiments, the formation of a recruited protein-molecule-target protein complex may be observed using mass spectrometric measurements or using Western blotting with various detection methods (e.g., staining, immunofluorescence, or radioactivity) to identify ternary complexes.

In some embodiments, the screening step comprises screening for a specific functional effect, such as activation, inhibition or inactivation, depending upon the nature of the screening assay. In certain embodiments, screening comprises identifying a change in activity of the target protein, as measured by the assay. In other embodiments, screening comprises identifying a change in the concentration of the target protein, for example, disappearance of the target protein (e.g., as a result of proteolysis).

### Plurality of Target Proteins for Screening

For the methods described herein that may be employed to identify or discover a target protein capable of being modulated by a known biological (e.g., recruited) entity, the methods of the present disclosure may further comprise screening each derivatized template molecule, and optionally also the template molecule, against a plurality of target proteins in the presence of the biological entity in order to determine whether any of the target proteins within the plurality of target proteins binds with the derivatized template molecule and biological entity to form a recruited entity-molecule-target protein complex. The plurality of target proteins against which the molecule-biological entity complexes are screened constitute a sample population of target proteins which could be identified as potential partners of the biological entity for entity-protein (e.g. protein-protein) interactions and may form a ternary complex with the molecule-biological entity complex. A “positive hit” in the screen would indicate potential formation of a recruited entity-molecule-target protein complex (e.g. a recruited protein-molecule-target protein complex). The recruited entity-molecule-target protein complex as used herein is referred to interchangeably with the terms “entity-molecule-protein complex”.

In some embodiments, the plurality of target proteins comprises a complete cellular proteome. In certain embodiments, the screening comprises a cell-based or lysate-based screen. In some embodiments, the plurality of target proteins includes the biological entity. In other embodiments, the plurality of target proteins excludes the biological entity. The screening as provided herein allows for the discovery of target protein interaction partners for the biological entity, such that the identity of the target protein partner need not be known prior to the screen and can be identified in subsequent work-up as a “target protein for the recruited entity”.

As described above, it should be recognized that the derivatized template molecules and biological entity may form ternary complexes with target proteins, wherein the biological entity may be a protein or other biological entity that may be present in cells or cell lysates, including but not limited to nucleic acids, organelles, and/or lipid membranes or fragments thereof. Such complexes may be referred to as “recruited entity-molecule-target protein complexes”, wherein the biological entity which engages the target protein and the derivatized template molecule is referred to as the “recruited entity”. It should be recognized that the term “ternary complex” may also be used to refer to a recruited protein-molecule-target protein complex, or protein-molecule-protein complex, or a recruited entity-molecule-target protein complex, if only one entity or protein is recruited by the molecule and target protein. It should be further recognized that more than one biological entity or protein may be recruited into interactions with the derivatized template molecule and target protein, which could result in a greater than three-way interaction between the two or more recruited entities, the derivatized template molecule and the target protein.

In some embodiments, the screening of the library of derivatized template molecules against a plurality of target proteins in the presence of the biological entity comprises contacting the library of derivatized template molecules with the plurality of target proteins in the presence of the biological entity. In some embodiments, the screening of the template molecule and library of derivatized template molecules against a plurality of target proteins in the presence of the biological entity comprises contacting the template molecule and library of derivatized template molecules with the plurality of target proteins in the presence of the biological entity.

In some embodiments, the screening of the library of derivatized template molecules against the plurality of target proteins in the presence of the biological entity is carried out in one step, wherein the library of derivatized template molecules are combined with the biological entity and the plurality of target proteins to be screened simultaneously. In other embodiments, the screening of the library of derivatized template molecules may be carried out in at least two sequential steps. For example, in some embodiments, the library of derivatized template molecules is first contacted with the biological entity to form a library of molecule-biological entity complexes. As noted above, in circumstances wherein the binding portion of the template molecule is not known, the derivatizing step could potentially reduce or eliminate the binding affinity of the derivatized template molecules for the biological entity. In some embodiments, at least one molecule-biological entity complex comprises at least one template molecule-biological entity complex.

In some embodiments, the screening of the template molecule and the library of derivatized template molecules against the plurality of target proteins in the presence of the biological entity is carried out in one step, wherein the template molecule and the library of derivatized template molecules are combined with the biological entity and the plurality of target proteins to be screened simultaneously. In other embodiments, the screening of the template molecule and the library of derivatized template molecules may be carried out in at least two sequential steps. For example, in some embodiments, the library of derivatized template molecules, and optionally also the template molecule, is first contacted with the biological entity to form a library of molecule-biological entity complexes. As noted above, in circumstances wherein the binding portion of the template molecule is not known, the derivatizing step could potentially reduce or eliminate the binding affinity of the derivatized template molecules for the biological entity. In some embodiments, at least one molecule-biological entity complex comprises at least one template molecule-biological entity complex.

The template molecule may also be contacted with the biological entity to form a template molecule-biological entity complex, which may then be employed as a control sample in evaluating the binding behavior of the derivatized template molecules. In other embodiments, at least one molecule-biological entity complex comprises at least one template molecule-biological entity complex.

Suitable screening assays may include but are not limited to assays such as, Western blotting, AlphaLISA, mass spectrometry-based screening, or screens that indicate modulation (such as inhibition, activation, or inactivation) or disappearance (e.g., degradation) of the target protein. In some embodiments, the step of screening comprises a high-throughput screen, wherein the library of molecule-target protein complexes is screened against the plurality of proteins in parallel. In some embodiments, the screening is carried out in one or more multi-well plates. In other embodiments wherein the derivatizing step is carried out in one or more multi-well plates, the screening is carried out in the same multi-well plates of the derivatizing step. In certain embodiments wherein the derivatizing step is carried out in one or more multi-well plates, the screening is carried out in the same multi-well plates of the derivatizing step without the need for purification of the library members prior to the screening step. In certain embodiments wherein the derivatizing step is carried out in one or more multi-well plates, some or all of the crude reaction mixture is transferred to one or more different multi-well plates for screening.

In some embodiments, the screening step comprises screening for formation of a recruited entity-molecule-target protein complex. In some embodiments, the screening step comprises screening for formation of a recruited protein-molecule-target protein complex. The formation of a recruited protein-molecule-target protein complex may be observed using distance- or proximity-dependent assays, such as time-resolved fluorescence resonance energy transfer (TR-FRET), AlphaLISA amplified luminescent proximity homogeneous assays, photoreactive cross-linking labeling assays, etc. In still other embodiments, the formation of a recruited protein-molecule-target protein complex may be observed using mass spectrometric measurements or using Western blotting with various detection methods (e.g., staining, immunofluorescence, or radioactivity) to identify ternary complexes.

In some embodiments, the screening step comprises screening for a specific functional effect, such as activation, inhibition or inactivation, depending upon the nature of the screening assay. In certain embodiments, screening comprises identifying a change in activity of the target protein, as measured by the assay. In other embodiments, screening comprises identifying a change in the concentration of the target protein, for example, disappearance of the target protein (e.g., as a result of proteolysis).

Designation, Identification and/or Measurement

For the methods described herein that may be employed to identify or discover a recruited entity capable of modulating a target protein, following the screening step, the methods of the present disclosure may comprise designating the template molecule or the derivatized template molecule in the recruited protein-molecule-target protein complex as a bridging molecule for the recruited protein and the target protein. The results of the screening step, which can provide evidence of the formation of a protein-molecule-protein complex, may or may not provide information regarding the identity of the recruited protein or the structure of the derivatized template molecule within the complex.

The designation of the bridging molecule (and also identification of the recruited protein) may be facilitated by the use of one or more reactive groups capable of forming a covalent bond to the target protein and/or the recruited protein, by providing a relative stable, long-lived ternary complex that may be isolated and purified. The reactive groups may be present in the bridging molecule, or they may be added specifically to facilitate target identification. In some embodiments, the template molecule or derivatized template molecule within the recruited protein-molecule-target protein complex is covalently bound to one or both of the target protein and the recruited protein. In certain embodiments, the template molecule or derivatized template molecule within the recruited protein-molecule-target protein complex is covalently bound to the target protein. In certain other embodiments, the template molecule or derivatized template molecule within the recruited protein-molecule-target protein complex is covalently bound to the recruited protein. In other embodiments, the template molecule or derivatized template molecule within the recruited protein-molecule-target protein complex is covalently bound to both of the target protein and the recruited protein.

However, even with the use of reactive groups as described above, the designation of a (derivatized) template molecule as a bridging molecule from a screen of recruited protein-molecule-target protein complexes may require steps such as deconvolution of combinatorial labeling and/or sample pooling, repetition of high-throughput synthesis of a subset of derivatized template molecules and/or high-throughput screens against a subset of the original plurality of proteins, as well as subsequent isolation and/or purification of the recruited protein-molecule-target protein complex(es). In some embodiments wherein the method comprises high-throughput synthesis and/or high-throughput screening, the method further comprises one or more additional iterations of the preceding steps to prepare a library of derivatized template molecules, and screening the library against a plurality of proteins. For example, in some embodiments, the designating step comprises re-synthesizing the tentative bridging molecule and preparing a library of derivatives of the tentative bridging molecule, and screening the tentative bridging molecule and library of derivatives against a plurality of proteins. In certain embodiments, the library of derivatives of the tentative bridging molecule may be prepared by introducing affinity tags, such as biotin or Click chemistry reactive alkynes and/or azides, to enable easier extraction and designation of the bridging molecule. In some embodiments, the designating step comprises isolation and/or purification of the protein-molecule-protein complex.

The template and/or derivatized template molecules that are found to induce formation of a protein-molecule-protein complex may be designated as bridging molecules, or a “molecular glues”. The designated bridging molecules may be subjected to additional engineering and derivatization, including according to the high-throughput methods as generally described above, in order to further optimize the molecular glue for promoting protein-protein interactions. In some embodiments of the present aspect, the derivatizing step, the screening step, identifying step, and designating step are repeated in sequence in one or more additional iterations. The number of additional iterations may be as few as one additional iteration or as many as several hundreds of additional iterations. In certain embodiments, the screening step, identifying step, and designating step are repeated in sequence in at least one additional iteration, at least 10 additional iterations, at least 50 additional iterations at least 100 additional iterations or at least 500 additional iterations. It should be recognized that the bridging molecule designated in one iteration may be used as the next “template molecule” to be derivatized in the next iteration, and so forth.

In still further embodiments, the method of the present aspect further comprises derivatizing the designated bridging molecule to form a library of derivatized bridging molecules; contacting the target protein with the library of derivatized bridging molecules to form at least one molecule-target protein complex, said at least one molecule-target protein complex comprising one or more derivatized bridging molecule-target protein complexes; screening the molecule-target protein complex against a plurality of proteins; optionally identifying one or more proteins in the plurality of proteins that interact with the molecule-target protein complex to form a recruited protein-molecule-target protein complex; and designating the derivatized bridging molecule in the recruited protein-molecule-target protein complex as a second bridging molecule for the recruited protein and the target protein.

In some embodiments, the methods provided herein further comprise identifying one or more proteins in the plurality of proteins that interact with the molecule-target protein complex to form a recruited protein-molecule-target protein complex. Similar to the designation of the bridging molecule above, the identifying step may similarly comprise deconvolution of combinatorial labeling and/or sample pooling, repetition of high-throughput synthesis of a subset of derivatized template molecules and/or high-throughput screens against a subset of the original plurality of proteins, as well as subsequent isolation and/or purification of the recruited protein-molecule-target protein complex(es), in order to determine the identity of the recruited protein.

In still further embodiments, once the recruited protein has been identified, the method optionally further comprises measuring an interaction of the recruited protein with the template molecule-target protein complex or with the derivatized template molecule-target protein complex to which it is bound.

In some embodiments, the interaction between the recruited protein and the target protein is modulation, binding affinity, inhibition, activation, phosphorylation, ubiquitination, acylation, inactivation, degradation, destabilization or unfolding. In some embodiments, the interaction to be measured between the recruited protein and the molecule-target protein complex is modulation, binding affinity, inhibition, activation, inactivation or degradation. In some embodiments wherein the screening step comprises screening for a functional effect, the measuring step comprises using the same assay as the assay used in the screening step. In certain embodiments wherein binding affinity is measured, the binding affinity is measured by determining a member selected from the group consisting of a biological activity of the protein, a conformational state of the protein, a dissociation constant of a test ligand for the protein, an affinity constant of a test ligand for the protein, a melting temperature of the protein, and a denaturing temperature for the protein.

While the above exemplary methods and embodiments are provided with regards to when the recruited entity is a recruited protein, it is to be understood that similar or analogous methods and embodiments may be used in the designation, identification, measurement, and/or optimization of recruited entities other than proteins.

While the below exemplary methods and embodiments are provided with regards to when the biological entity comprises a protein, it is to be understood that similar or analogous methods and embodiments may be used in the designation, identification, measurement, and/or optimization procedures when the recruited biological entity comprises entities other than proteins.

For the methods described herein that may be employed to identify or discover a target protein capable of being modulated by a known biological (e.g., recruited) entity, following the screening step, the methods of the present disclosure may comprise designating the template molecule or the derivatized template molecule in the recruited protein-molecule-target protein complex as a bridging molecule for the recruited protein and the target protein. The results of the screening step, which can provide evidence of the formation of a protein-molecule-protein complex, may or may not provide information regarding the identity of the target protein or the structure of the derivatized template molecule within the complex.

The designation of the bridging molecule (and also identification of the target protein) may be facilitated by the use of one or more reactive groups capable of forming a covalent bond to the target protein and/or the recruited protein, by providing a relative stable, long-lived ternary complex that may be isolated and purified. The reactive groups may be present in the bridging molecule, or they may be added specifically to facilitate target identification. In some embodiments, the template molecule or derivatized template molecule within the recruited protein-molecule-target protein complex is covalently bound to one or both of the target protein and the recruited protein. In certain embodiments, the template molecule or derivatized template molecule within the recruited protein-molecule-target protein complex is covalently bound to the target protein. In certain other embodiments, the template molecule or derivatized template molecule within the recruited protein-molecule-target protein complex is covalently bound to the recruited protein. In other embodiments, the template molecule or derivatized template molecule within the recruited protein-molecule-target protein complex is covalently bound to both of the target protein and the recruited protein.

However, even with the use of reactive groups as described above, the designation of a (derivatized) template molecule as a bridging molecule from a screen of recruited protein-molecule-target protein complexes may require steps such as deconvolution of combinatorial labeling and/or sample pooling, repetition of high-throughput synthesis of a subset of derivatized template molecules and/or high-throughput screens against a subset of the original plurality of target proteins, as well as subsequent isolation and/or purification of the recruited protein-molecule-target protein complex(es). In some embodiments wherein the method comprises high-throughput synthesis and/or high-throughput screening, the method further comprises one or more additional iterations of the preceding steps to prepare a library of derivatized template molecules, and screening the library against a plurality of target proteins. For example, in some embodiments, the designating step comprises re-synthesizing the tentative bridging molecule and preparing a library of derivatives of the tentative bridging molecule, and screening the tentative bridging molecule and library of derivatives against a plurality of target proteins. In certain embodiments, the library of derivatives of the tentative bridging molecule may be prepared by introducing affinity tags, such as biotin or Click chemistry reactive alkynes and/or azides, to enable easier extraction and designation of the bridging molecule. In some embodiments, the designating step comprises isolation and/or purification of the protein-molecule-protein complex.

The template and/or derivatized template molecules that are found to induce formation of a protein-molecule-protein complex may be designated as bridging molecules, or as “molecular glues”. The designated bridging molecules may be subjected to additional engineering and derivatization, including according to the high-throughput methods as generally described above, in order to further optimize the molecular glue for promoting protein-protein interactions. In some embodiments of the present aspect, the derivatizing step, the screening step, identifying step, and designating step are repeated in sequence in one or more additional iterations. The number of additional iterations may be as few as one additional iteration or as many as several hundreds of additional iterations. In certain embodiments, the screening step, identifying step, and designating step are repeated in sequence in at least one additional iteration, at least 10 additional iterations, at least 50 additional iterations at least 100 additional iterations or at least 500 additional iterations. It should be recognized that the bridging molecule designated in one iteration may be used as the next “template molecule” to be derivatized in the next iteration, and so forth.

In still further embodiments, the method of the present aspect further comprises derivatizing the designated bridging molecule to form a library of derivatized bridging molecules; contacting the biological entity with the library of derivatized bridging molecules to form at least one molecule-biological entity complex, said at least one molecule-biological entity complex comprising one or more derivatized bridging molecule-biological entity complexes; screening the molecule-biological entity complex against a plurality of target proteins; optionally identifying one or more target proteins in the plurality of target proteins that interact with the molecule-biological entity complex to form a recruited protein-molecule-target protein complex; and designating the derivatized bridging molecule in the recruited protein-molecule-target protein complex as a second bridging molecule for the recruited protein and the target protein.

In some embodiments, the methods provided herein further comprise identifying one or more target proteins in the plurality of target proteins that interact with the molecule-biological entity complex to form a recruited protein-molecule-target protein complex. Similar to the designation of the bridging molecule above, the identifying step may similarly comprise deconvolution of combinatorial labeling and/or sample pooling, repetition of high-throughput synthesis of a subset of derivatized template molecules and/or high-throughput screens against a subset of the original plurality of target proteins, as well as subsequent isolation and/or purification of the recruited protein-molecule-target protein complex(es), in order to determine the identity of the target protein.

In still further embodiments, once the target protein has been identified, the method optionally further comprises measuring an interaction of the target protein with the template molecule-recruited protein complex or with the derivatized template molecule-recruited protein complex to which it is bound.

In some embodiments, the interaction between the recruited protein and the target protein is modulation, binding affinity, inhibition, activation, phosphorylation, ubiquitination, acylation, inactivation, degradation, destabilization, or unfolding. In some embodiments, the interaction to be measured between the target protein and the molecule-biological entity complex is modulation, binding affinity, inhibition, activation, inactivation or degradation. In some embodiments wherein the screening step comprises screening for a functional effect, the measuring step comprises using the same assay as the assay used in the screening step. In certain embodiments wherein binding affinity is measured, the binding affinity is measured by determining a member selected from the group consisting of a biological activity of the protein, a conformational state of the protein, a dissociation constant of a test ligand for the protein, an affinity constant of a test ligand for the protein, a melting temperature of the protein, and a denaturing temperature for the protein.

### III. Use of Designated Bridging Molecules

In one aspect, provided herein are bridging molecules obtained according to the methods described herein. In another aspect, the present disclosure provides methods for use of bridging molecules obtained according to the methods described herein.

The bridging molecules as provided herein may be subsequently utilized for modulation of the target protein in vitro, in vivo, and/or in silico. In particular, for bridging molecules found to promote protein-protein interactions for an oncogenic target protein, the bridging molecules may find utility in treatment of hyperproliferative disorders associated with the target protein.

In yet another aspect, provided herein are methods for modulating the activity of a target protein, comprising contacting the target protein with a bridging molecule in the presence of a recruited protein, wherein the target protein, bridging molecule and recruited protein form a recruited protein-molecule-target protein complex. In some embodiments of the present aspect, the recruited protein induces inhibition, inactivation, activation or degradation of the target protein. In some embodiments, the inhibition, inactivation, activation or degradation of the target protein is induced by the interaction between the target protein and the recruited protein. In some embodiments wherein the degradation of the target protein is induced by the recruited protein, the recruited protein is an E3 ligase. In still other embodiments, the inhibition, inactivation, activation or degradation of the target protein is induced by the bridging molecule. In still other embodiments, the bridging molecule induces the degradation of the target protein by causing the target protein to unfold.

In yet another aspect, provided herein are methods for modulating the activity of a target protein, comprising contacting the target protein with a bridging molecule in the presence of a recruited entity, wherein the target protein, bridging molecule and recruited entity form a recruited entity-molecule-target protein complex. In some embodiments of the present aspect, the recruited entity induces inhibition, inactivation, activation or degradation of the target protein. In some embodiments, the inhibition, inactivation, activation or degradation of the target protein is induced by the interaction between the target protein and the recruited entity. In some embodiments wherein the degradation of the target protein is induced by the recruited entity, the recruited entity is an E3 ligase. In still other embodiments, the inhibition, inactivation, activation or degradation of the target protein is induced by the bridging molecule. In still other embodiments, the bridging molecule induces the degradation of the target protein by causing the target protein to unfold.

In still yet another aspect, provided herein are methods for treating or preventing a disease or disorder, such as hyperproliferative disorders, wherein the disease or disorder is mediated by a target protein. Disorders which are known to be associated or mediated by certain target proteins or particular signaling pathways may be suitable candidates for treatment with bridging molecules in order to induce protein-protein interactions involving the target protein and regulate the target protein's activity. In some embodiments, the method comprises contacting the target protein with a bridging molecule in the presence of a recruited protein, wherein the target protein, bridging molecule and recruited protein form a recruited protein-molecule-target protein complex. In some embodiments, the contacting is performed in vitro. In other embodiments, the contacting is performed in vivo. In still other embodiments, the contacting is performed in silico.

In still yet another aspect, provided herein are methods for treating or preventing a disease or disorder, such as hyperproliferative disorders, wherein the disease or disorder is mediated by a target protein. Disorders which are known to be associated or mediated by certain target proteins or particular signaling pathways may be suitable candidates for treatment with bridging molecules in order to induce entity-protein or protein-protein interactions involving the biological entity and regulate the target protein's activity. In some embodiments, the method comprises contacting the target protein with a bridging molecule in the presence of a recruited protein, wherein the target protein, bridging molecule and recruited protein form a recruited protein-molecule-target protein complex. In some embodiments, the contacting is performed in vitro. In other embodiments, the contacting is performed in vivo. In still other embodiments, the contacting is performed in silico.

In some embodiments, the target protein is a kinase. In some embodiments, the target protein is selected from the group consisting of: Ras proteins, RAF kinases, and cyclin dependent kinases. In some embodiments, the target protein is a Ras protein. In certain embodiments, the target protein is K-Ras, H-Ras, or N-Ras. In other embodiments, the target protein is a RAF kinase. In certain variations, the RAF kinase is A-Raf, B-Raf, or C-Raf. In other embodiments, the target protein is a cyclin dependent kinase (CDK). In certain other variations, the cyclin dependent kinase is CDK1, CDK2, CDK3, CDK4, CDK5, CDK6, CDK7, CDK8, CDK9, CDK10, CDK11, CDK12, CDK13, CDK14, CDK15, CDK16, CDK17, CDK18, CDK19, or CDK20.

### EXAMPLES

The presently disclosed subject matter will be better understood by reference to the following Examples, which are provided as exemplary of the invention, and not by way of limitation.

### Example 1

The present example describes an exemplary method for the preparation of a derivatized template molecule library and high-throughput screen to identify bridging molecules for creating a ternary complex of the bridging molecule and two proteins. The present example generally follows the process as described in FIG. 1.

A target protein and a known ligand or binding fragment for the target protein are selected. The ligand is taken to serve as a template molecule for further derivatization in order to prepare a compound library of derivatives. The template molecule is analyzed to identify functional groups present on the template molecule that are suitable for derivatization, such as to add covalently binding reactive groups (“warheads”) or other structural moieties. Computational analysis is optionally used to design and analyze prospective chemical libraries for sufficient structural diversity and also to develop a strategic approach for preparing the prospective chemical library using high-throughput synthesis methods (e.g., divergent synthesis, combinatorial chemistry). If the moiety or region of the template molecule that binds to the target protein is known, then this moiety or region may be excluded from the functional groups to be derivatized and high-throughput synthetic methods may be adapted to avoid unwanted reactions with the binding moiety.

The compound library of derivatized molecules is synthesized. The library of derivatized molecules is combined with the target protein to form a library of derivatized molecule-target protein complexes. The library of the derivatized molecule-target protein complexes is screened against various test proteins using high-throughput screening assays to screen for formation of protein-molecule complexes, such as AlphaLISA, or to screen for changes in target protein function, such as inhibition assays or degradation assays. A positive hit from the screening assay indicates the formation of a test protein-derivatized molecule-target protein complex.

The test protein-derivatized molecule-target protein complex is optionally identified, isolated and/or purified in order to determine the identity of the test protein and/or the derivatized molecule present in the ternary complex. The derivatized molecule is designated as a molecular glue.

Once the identity of the test protein has been determined, further evaluation of the protein-protein interaction between the target protein and the test protein is conducted. The test protein-derivatized molecule-target protein complex is prepared in vitro, and one or more assays (e.g., assays known in the art) are carried out on the test protein-derivatized molecule-target protein complex to measure any modulation of the target protein activity or concentration, including activation, inhibition, inactivation, or degradation.

### Example 2

The present example describes an exemplary method for the preparation of a derivatized template molecule library and high-throughput screen to identify bridging molecules for creating a ternary complex of the bridging molecule and two proteins (or between a biological entity and a protein). The present example generally follows the process as described in FIG. 2.

A biological entity and a known ligand or binding fragment for the biological entity are selected. The ligand is taken to serve as a template molecule for further derivatization in order to prepare a compound library of derivatives. The template molecule is analyzed to identify functional groups present on the template molecule that are suitable for derivatization, such as to add covalently binding reactive groups (“warheads”) or other structural moieties. Computational analysis is optionally used to design and analyze prospective chemical libraries for sufficient structural diversity and also to develop a strategic approach for preparing the prospective chemical library using high-throughput synthesis methods (e.g., divergent synthesis, combinatorial chemistry). If the moiety or region of the template molecule that binds to the biological entity is known, then this moiety or region may be excluded from the functional groups to be derivatized and high-throughput synthetic methods may be adapted to avoid unwanted reactions with the binding moiety.

The compound library of derivatized molecules is synthesized. The library of derivatized molecules is combined with the biological entity to form a library of derivatized molecule-biological entity complexes. The library of the derivatized molecule-biological entity complexes is screened against various test target proteins using high-throughput screening assays to screen for formation of protein-molecule complexes, such as AlphaLISA, or to screen for changes in target protein function, such as inhibition assays or degradation assays. A positive hit from the screening assay indicates the formation of a test target protein-derivatized molecule-biological entity complex.

The test target protein-derivatized molecule-biological entity complex is identified, isolated and/or purified in order to determine the identity of the test target protein and/or the derivatized molecule present in the ternary complex. The derivatized molecule is designated as a molecular glue.

Once the identity of the test target protein has been determined, further evaluation of the entity-protein (e.g. protein-protein) interaction between the biological entity and the test target protein is conducted. The test target protein-derivatized molecule-biological entity complex is prepared in vitro, and one or more assays (e.g., assays known in the art) are carried out on the test target protein-derivatized molecule-biological entity complex to measure any modulation of the target protein activity or concentration, including activation, inhibition, inactivation, or degradation.

### Example 3

The present example describes an exemplary method for high-throughput screening of a library of derivatized molecules to screen for changes in target protein function via a degradation assay. In this example, the target protein is a V600E mutant of B-Raf. The derivatized molecules are based on molecules known to bind to mutant B-Raf. A positive hit from the screening assay indicates represents degradation of the target protein by any of the aforementioned mechanisms.

Nano-Glo®HiBiT Lytic Assay (Promega, Madison, WI, USA) with a multi-well plate is chosen herein as an exemplary degradation assay. An A375.10 cell line is generated from an A375 cell line by knocking-in a HiBiT tag at the N-terminus of B-RafV600E protein via CRISPR technology.

Prior to the assay, the A375.10 cell line is maintained in DMEM no-phenol red medium supplemented with 10% fetal bovine serum (FBS). Following treatment with a derivatized molecule, B-RafV600E degradation is determined based on quantification of HiBiT luminescence signal by lysing the cells followed by addition of Nano-Glo® HiBiT Lytic Assay Reagents. The luminescence signal detected correlates with the total B-RafV600E protein level in cells. Briefly, derivatized molecules are added to the multi-well plate and diluted to an appropriate concentration. Then, a suspension of A375.10 cells is dispensed into the wells of the multi-well at an appropriate cell density. The plates are kept at 37° C. with 5% CO₂ for the duration of the assay. After the desired incubation time with compound, Nano-Glo® HiBiT Lytic Buffer containing LgBiT protein and luminescence substrate are added to the cells of the assay plate. The plate is then incubated at room temperature. Finally, HiBiT luminescence signal is acquired.

Quantification of luminescence responses measured in the presence of derivatized molecule are normalized to a high signal/no degradation control (untreated cells and lytic detection reagent) and a low signal/full degradation control (untreated cells, no lytic detection reagent). Data are analyzed with a 4-parameter logistic fit to generate sigmoidal dose-response curves. Parameters that can be used to describe the extent of B-RafV600E include DC₅₀, which is the concentration of compound at which exactly 50% of the total cellular B-Raf^{V600E} has been degraded, and Eₘₐₓ, or the maximum effect of each compound, which represents the amount of residual protein remaining in the cell following compound treatment. After this quantification analysis, the extent to which the function of the target protein has been changed can be compared and effective bridging molecules that function as target protein-bridging molecule-biological entity complexes can be identified, designated, and used as described.

What is claimed is:

1. A method of identifying a bridging molecule that creates a recruited entity-molecule-target protein complex, comprising:
selecting a target protein;
selecting a template molecule known or predicted to bind to the target protein, said template molecule comprising one or more functional groups;
derivatizing at least one of the one or more functional groups of the template molecule to form a library of derivatized template molecules; and
screening the library of derivatized template molecules against a plurality of biological entities in the presence of the target protein.

2. The method of claim 1, wherein the template molecule is known to bind to the target protein.

3. The method of claim 1 or 2, wherein the screening step comprises screening the template molecule and the library of derivatized template molecules against a plurality of biological entities in the presence of the target protein.

4. The method of any one of claims 1-3, wherein at least one of the template molecule, if present, and the derivatized template molecules forms a recruited entity-molecule-target protein complex with the target protein and at least one or more entities in the plurality of biological entities.

5. The method of claim 4, wherein the method further comprises designating the template molecule, if present, or the derivatized template molecule in the recruited entity-molecule-target protein complex as a bridging molecule for the recruited entity and the target protein.

6. The method of any one of claims 1-5, wherein the screening step comprises contacting the template molecule, if present, and the library of derivatized template molecules with the plurality of biological entities in the presence of the target protein.

7. The method of any one of claims 1-6, wherein the template molecule further comprises a reactive group wherein the reactive group is not derivatized during library formation and wherein the reactive group is capable of forming a covalent bond with the target protein and/or recruited entity.

8. The method of any one of claims 1-7, wherein the derivatized template molecule comprises a reactive group, wherein the reactive group is capable of forming a covalent bond with the target protein and/or the recruited entity.

9. The method of any one of claims 1-8, wherein the recruited entity is a recruited protein.

10. A method of identifying a bridging molecule that creates a recruited entity-molecule-target protein complex, comprising:
selecting a biological entity;
selecting a template molecule known or predicted to bind to the biological entity, said template molecule comprising one or more functional groups;
derivatizing at least one of the one or more functional groups of the template molecule to form a library of derivatized template molecules; and
screening the library of derivatized template molecules against a plurality of target proteins in the presence of the biological entity.

11. The method of claim 10, wherein the template molecule is known to bind to the biological entity.

12. The method of claim 10 or 11, wherein the screening step comprises screening the template molecule and the library of derivatized template molecules against a plurality of target proteins in the presence of the biological entity.

13. The method of any one of claims 10-12, wherein at least one of the template molecule, if present, and the derivatized template molecules forms a recruited entity-molecule-target protein complex with the biological entity and at least one or more target proteins in the plurality of target proteins.

14. The method of claim 13, wherein the method further comprises designating the template molecule, if present, or the derivatized template molecule in the recruited entity-molecule-target protein complex as a bridging molecule for the recruited entity and the target protein.

15. The method of any one of claims 10-14, wherein the screening step comprises contacting the template molecule, if present, and the library of derivatized template molecules with the plurality of target proteins in the presence of the biological entity.

16. The method of any one of claims 10-15, wherein the template molecule further comprises a reactive group wherein the reactive group is not derivatized during library formation and wherein the reactive group is capable of forming a covalent bond with the target protein and/or recruited entity.

17. The method of any one of claims 10-16, wherein the derivatized template molecule comprises a reactive group, wherein the reactive group is capable of forming a covalent bond with the target protein and/or the recruited entity.

18. The method of any one of claims 10-17, wherein the recruited entity is a recruited protein.

19. A method of identifying a bridging molecule that creates a recruited protein-molecule-target protein complex, comprising:
selecting a target protein;
selecting a template molecule known or predicted to bind to the target protein, said template molecule comprising one or more functional groups;
derivatizing at least one of the one or more functional groups of the template molecule to form a library of derivatized template molecules; and
screening the library of derivatized template molecules against a plurality of proteins in the presence of the target protein.

20. The method of claim 19, wherein the template molecule is known to bind to the target protein.

21. The method of claim 19 or 20, wherein the screening step comprises screening the template molecule and the library of derivatized template molecules against a plurality of biological entities in the presence of the target protein.

22. The method of any one of claims 19-21, wherein at least one of the template molecule, if present, and the derivatized template molecules forms a recruited protein-molecule-target protein complex with the target protein and at least one or more proteins in the plurality of proteins.

23. The method of claim 22, further comprising designating the template molecule, if present, or the derivatized template molecule in the recruited protein-molecule-target protein complex as a bridging molecule for the recruited protein and the target protein.

24. The method of any one of claims 19-23, wherein the screening step comprises contacting the template molecule, if present, and the library of derivatized template molecules with the plurality of proteins in the presence of the target protein.

25. The method of any one of claims 19-24, wherein the screening step comprises:
contacting the target protein with the template molecule, if present, and the library of derivatized template molecules to form at least one molecule-target protein complex, said at least one molecule-target protein complex comprising at least one of a template molecule-target protein complex or one or more derivatized template molecule-target protein complexes; and
screening the at least one molecule-target protein complex against a plurality of proteins, wherein the at least one molecule-target protein complex forms a recruited protein-molecule-target protein complex with at least one or more proteins in the plurality of proteins.

26. The method of any one of claims 19-25, wherein the template molecule further comprises a reactive group wherein the reactive group is not derivatized during library formation and wherein the reactive group is capable of forming a covalent bond with the target protein.

27. The method of any one of claims 19-26, wherein the template molecule further comprises a reactive group wherein the reactive group is not derivatized during library formation and wherein the reactive group is capable of forming a covalent bond with the recruited protein.

28. The method of any one of claims 19-27, wherein the derivatized template molecule comprises a reactive group wherein the reactive group is capable of forming a covalent bond with the target protein and/or the recruited protein.

29. The method of any one of claims 19-28, wherein said plurality of proteins excludes the target protein.

30. The method of any one of claims 1-9 and 19-29, wherein the portion of the template molecule which binds to the target protein is known, and the derivatizing of at least one of the one or more functional groups of the template molecule to form a library of derivatized template molecules is performed on functional groups of the template molecule which do not bind to the target protein.

31. The method of any one of claims 7-8 and 26-28, wherein the reactive group forms a covalent bond to one or more amino acid residue Cys, Lys, Ser, Tyr, His, Trp, Met, Asp, Glu or Thr on the target protein.

32. The method of any one of claims 19-31, further comprising identifying one or more proteins in the plurality of proteins that interact with the template molecule, if present, or derivatized template molecule and target protein to form a recruited protein-molecule-target protein complex.

33. A method of identifying a bridging molecule that creates a recruited protein-molecule-target protein complex, comprising:
selecting a biological entity comprising a protein;
selecting a template molecule known or predicted to bind to the biological entity, said template molecule comprising one or more functional groups;
derivatizing at least one of the one or more functional groups of the template molecule to form a library of derivatized template molecules; and
screening the library of derivatized template molecules against a plurality of target proteins in the presence of the biological entity.

34. The method of claim 33, wherein the template molecule is known to bind to the biological entity.

35. The method of claim 33 or 34, wherein the screening step comprises screening the template molecule and the library of derivatized template molecules against a plurality of target proteins in the presence of the biological entity.

36. The method of any one of claims 33-35, wherein at least one of the template molecule, if present, and the derivatized template molecules forms a recruited protein-molecule-target protein complex with the biological entity and at least one or more target proteins in the plurality of target proteins.

37. The method of claim 36, further comprising designating the template molecule, if present, or the derivatized template molecule in the recruited protein-molecule-target protein complex as a bridging molecule for the recruited protein and the target protein.

38. The method of any one of claims 33-37, wherein the screening step comprises contacting the template molecule, if present, and the library of derivatized template molecules with the plurality of target proteins in the presence of the biological entity.

39. The method of any one of claims 3-38, wherein the screening step comprises:
contacting the biological entity with the template molecule, if present, and the library of derivatized template molecules to form at least one molecule-biological entity complex, said at least one molecule-biological entity complex comprising at least one of a template molecule-biological entity complex or one or more derivatized template molecule-biological entity complexes; and
screening the at least one molecule-biological entity complex against a plurality of target proteins, wherein the at least one molecule-biological entity complex forms a recruited protein-molecule-target protein complex with at least one or more target proteins in the plurality of target proteins.

40. The method of any one of claims 33-39, wherein the template molecule further comprises a reactive group wherein the reactive group is not derivatized during library formation and wherein the reactive group is capable of forming a covalent bond with the biological entity.

41. The method of any one of claims 33-40, wherein the template molecule further comprises a reactive group wherein the reactive group is not derivatized during library formation and wherein the reactive group is capable of forming a covalent bond with the target protein.

42. The method of any one of claims 33-41, wherein the derivatized template molecule comprises a reactive group wherein the reactive group is capable of forming a covalent bond with the biological entity and/or the target protein.

43. The method of any one of claims 33-42, wherein said plurality of target proteins excludes the biological entity.

44. The method of any one of claims 10-18 and 33-43, wherein the portion of the template molecule which binds to the biological entity is known, and the derivatizing of at least one of the one or more functional groups of the template molecule to form a library of derivatized template molecules is performed on functional groups of the template molecule which do not bind to the biological entity.

45. The method of any one of claims 16-17 and 40-42, wherein the reactive group forms a covalent bond to one or more amino acid residue Cys, Lys, Ser, Tyr, His, Trp, Met, Asp, Glu or Thr on the biological entity.

46. The method of any one of claims 33-45, further comprising identifying one or more target proteins in the plurality of target proteins that interact with the template molecule, if present, or derivatized template molecule and biological entity to form a recruited protein-molecule-target protein complex.

47. The method of any one of claims 10-18 and 33-46, wherein the biological entity is a protein involved in degradation target proteins.

48. The method of claim 47, wherein the biological entity is an adaptor protein or an autophagy protein.

49. The method of claim 47, wherein the biological entity is an E3 ligase.

50. The method of claim 47, wherein the biological entity is an E2 ligase.

51. The method of claim 47, wherein the biological entity is selected from the group consisting of VHL, cereblon, MDM2, an IAP, and a DCAF.

52. The method of claim 47, wherein the biological entity is selected from the group consisting of KEAP1, AHR, BIRC3, RNF4, RNF114, RNF43, RNF7, RNF130, DCAF4, DCAF1, DCAF11, XIAP, and cIAP.

53. The method of any one of claims 1-52, wherein the screening of the template molecule, if present, and the library of derivatized template molecules is performed using a high-throughput screen.

54. The method of any one of claims 1-53, wherein the screening of the template molecule, if present, and the library of derivatized template molecules is performed using AlphaELISA, mass spectrometry-based screening, Western blotting, photoreactive cross-linking, photoreactive cross-linking labeling assay, inhibition assays, activation assays, degradation assays, or concentration assays.

55. The method of claim 54, wherein the screening is performed using a degradation assay based on luminescence, fluorescence, or Western blotting.

56. The method of any one of claims 1-55, wherein the screening of the template molecule, if present, and the library of derivatized template molecules is performed using a cell- or lysate-based assay.

57. The method of claim 56, wherein the screening step is performed using a cell-based assay.

58. The method of claim 56, wherein the screening step is performed using a cell lysate-based assay.

59. The method of any one of claims 1-58, wherein the one or more functional groups of the template molecule comprises one or more of an alkyl, alkenyl, alkynyl, vinyl, allyl, halide, haloalkyl, hydroxyl, alkoxy, ether, thiol, thioether, disulfide, sulfoxide, sulfone, sulfinic acid, sulfonic acid, sulfonate ester, carbonyl, carboxylic acid, anhydride, acyl halide, aryl halide, ester, aldehyde, carbonate, carbamoyl, acetal, ketal, amino, amido, carboxamido, imino, imido, nitro, nitrate, nitrite, nitroso, azido, cyano, cyanato, isocyanato, thiocyanato, isothocyanato, sulfonyl, azo, epoxide, peroxide, phenyl, phenol, aryl, heteroaryl, cycloalkyl, cycloalkenyl, cycloalkynyl, heterocycloalkyl, heterocycloalkenyl, heterocycloalkynyl, heteroalkyl, phosphate, phosphine, boronic acid, boronic acid ester, or silylether.

60. The method of any one of claims 1-59, wherein the step of derivatizing comprises nucleophilic substitution, nucleophilic aromatic substitution, electrophilic substitution, addition, elimination, acylation, esterification, amidation, amination oxidation, reduction, cyclization, cross-coupling or rearrangement of at least one of the one or more functional groups.

61. The method of any one of claims 1-60, wherein the one or more functional groups is derivatized by chemical groups that contain chiral centers, sterically rigid moieties (e.g., phenyl ring) or sterically flexible moieties (e.g., linear alkyls), hydrogen bond donors or acceptors, polarizable or non-polarizable moieties (e.g., soft versus hard).

62. The method of any one of claims 7-8, 16-17, 26-28, 31, 40-42 and 45, wherein the reactive group comprises one or more of a halo, amino, thiol, disulfide, thiirane, aziridine, alkenyl, alkynyl, ester, sulfonic acid ester, thioester, N-hydroxysucinnimidyl ester, α,β-unsaturated keto ester, acrylate, (cyano) acrylamide, epoxide, vinyl sulfone, vinyl sulfonamide, aldehyde, ketone, nitrile, sulfonyl fluoride, fluorosulfate, squaric acid derivative, propiolamide, butynamide, urea, carbamate, lactone, chlorofluoroacetamide, or beta-lactam.

63. The method of any one of claims 7-8, 16-17, 26-28, 31, 40-42 and 45, wherein the reactive group comprises one or more of a halo, amino, thiol, disulfide, thiirane, aziridine, alkenyl, alkynyl, ester, sulfonic acid ester, thioester, N-hydroxysucinnimidyl ester, α,β-unsaturated keto ester, acrylate, (cyano) acrylamide, epoxide, vinyl sulfone, vinyl sulfonamide, aldehyde, ketone, nitrile, sulfonyl fluoride, fluorosulfate, squaric acid derivative, propiolamide, butynamide, or beta-lactam.

64. The method of any one of claims 7-8, 16-17, 26-28, 31, 40-42, 45, and 62-63 and 53, wherein the covalent bond is irreversible.

65. The method of any one of claims 7-8, 16-17, 26-28, 31, 40-42, 45, and 62-63, wherein the covalent bond is reversible.

66. The method of any one of claims 1-65, wherein the derivatizing step is carried out in one or more multi-well plates.

67. The method of claim 65, wherein the screening is carried out in the same multi-well plates of the derivatizing step.

68. The method of claim 65, wherein the derivatizing step is carried out in one or more multi-well plates, some or all of the crude reaction mixture is transferred to one or more different multi-well plates for screening.

69. The method of any one of claims 1-68, wherein the derivatizing step is performed without purification prior to the screening step.

70. The method of any one of claims 19-69, further comprising measuring an interaction of the recruited protein with the target protein.

71. The method of claim 70, wherein the interaction between the recruited protein and the target protein is modulation, binding affinity, inhibition, activation, phosphorylation, ubiquitination, acylation, inactivation, degradation, destabilization or unfolding.

72. The method of claim 71, wherein the binding affinity is measured by determining a member selected from the group consisting of a biological activity of the protein, a conformational state of the protein, a dissociation constant of a test ligand for the protein, an affinity constant of a test ligand for the protein, a melting temperature of the protein, and a denaturing temperature for the protein.

73. A bridging molecule as obtained from the method according to claims 1-72.

74. A method for modulating the activity of a target protein, comprising: contacting the target protein with a bridging molecule in the presence of a recruited protein, wherein the target protein, bridging molecule and recruited protein form a recruited protein-molecule-target protein complex.

75. The method of claim 74, wherein the recruited protein induces inhibition, inactivation, activation or degradation of the target protein.

76. A method for modulating the activity of a target protein, comprising: contacting the target protein with a bridging molecule in the presence of a recruited entity, wherein the target protein, bridging molecule and recruited entity form a recruited entity-molecule-target protein complex.

77. The method of claim 76, wherein the recruited entity induces inhibition, inactivation, activation or degradation of the target protein.

78. The method of any one of claims 73-77, wherein the contacting step is performed in vitro.

79. The method of any one of claims 73-77, wherein the contacting step is performed in vivo.

80. The method of any one of claims 73-77, wherein the contacting step is performed in silico.

81. The method of any one of claims 1-71 and 73-80, wherein the target protein is a Ras protein.

82. The method of claim 81, wherein the target protein is K-Ras, H-Ras, or N-Ras.

83. The method of any one of claims 1-72 and 74-80, wherein the target protein is a kinase.

84. The method of claim 83, wherein the kinase is a RAF kinase or a cyclin dependent kinase (CDK).

85. The method of claim 84, wherein the RAF kinase is A-Raf, B-Raf, or C-Raf.

86. The method of claim 84, wherein the cyclin dependent kinase is CDK1, CDK2, CDK3, CDK4, CDK5, CDK6, CDK7, CDK8, CDK9, CDK10, CDK11, CDK12, CDK13, CDK14, CDK15, CDK16, CDK17, CDK18, CDK19, or CDK20.

87. The method of any one of claims 76-86, wherein the biological entity is a protein involved in degradation of target proteins.

88. The method of claim 87, wherein the biological entity is an adaptor protein or an autophagy protein.

89. The method of claim 87, wherein the biological entity is an E3 ligase.

90. The method of claim 87, wherein the biological entity is an E2 ligase.

91. The method of claim 87, wherein the biological entity is selected from the group consisting of VHL, cereblon, MDM2, an IAP, and a DCAF.

92. The method of claim 87, wherein the biological entity is selected from the group consisting of KEAP1, AHR, BIRC3, RNF4, RNF114, RNF43, RNF7, RNF130, DCAF4, DCAF1, DCAF11, XIAP, and cIAP.
